(19) United States
(12) Patent Application Publication
(10) Pub. No.: US 2004/0258660 A1
(43) Pub. Date: Dec. 23, 2004

(54) Immunogenic mimetics of multimer proteins with promiscuous T cell epitope inserts

(76) Inventors: Steen Klysner, Horsholm (DK); Finn Stausholm Nielsen, Horsholm (DK); Tomas Bratt, Horsholm (DK); Bjorn Voldborg, Horsholm (DK); Soren Mouritsen, Horsholm (DK)

Correspondence Address: FROMMER LAWRENCE & HAUG, 745 FIFTH AVENUE- 10TH FL., NEW YORK, NY, 10151, US

(21) Appl. No.: 10/846,911

(22) Filed: May 14, 2004

Related U.S. Application Data
(63) Continuation-in-part of application No. PCT/DK02/00764, filed on Nov. 15, 2002.
(60) Provisional application No. 60/331,575, filed on Nov. 16, 2001.

(30) Foreign Application Priority Data
Nov. 16, 2001 (DK) PA 2001 01702

(51) Int. Cl. C07K 14/525; C07H 21/04; C12P 21/02; A61K 38/19

(52) U.S. Cl. 424/85.1; 435/69.5; 435/320.1; 435/325; 530/351; 536/23.5

(57) ABSTRACT

The present invention relatas to novel immunogenic variants of multimeric proteins such as immunogenic variants of interleukin 5 (IL5) and tumour necrosis factor alpha (TNF, TNFα). The variants are, besides from being immunogenic in the autologous host, also highly similar to the native 3D structure of the proteins from which they are derived. Certain variants are monomeric mimics of the multimers, where peptide linkers (inert or T helper epitope containing) ensure a spatial organisation of the monomomer units that facilitate correct folding. A subset of variants are monomer TNFα variants that exhibit a superior capability of assembling into multimers with a high structural similarity to the native protein. Also disclosed are methods of treatment and production of the variants as well as DNA fragments, vectors, and host cells.

### REFERENCE TO RELATED APPLICATIONS

This application is a continuation-in-part of International Patent Application PCT/DK02/00764 filed Nov. 15, 2002 and published as WO 03/042244 on May 22, 2003, which claims priority to Danish Patent Application 2001 01702 filed Nov. 16, 2001 and U.S. Provisional Patent Application Ser. No. 60/331,575 filed Nov. 16, 2001.

Each of the applications and patents mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

It is noted that in this disclosure, terms such as “comprises”, “comprised”, “comprising”, “contains”, “containing” and the like can have the meaning attributed to them in U.S. Patent law; e.g., they can mean “includes”, “included”, “including” and the like. Terms such as “consisting essentially of” and “consists essentially of” have the meaning attributed to them in U.S. Patent law, e.g., they allow for the inclusion of additional ingredients or steps that do not detract from the novel or basic characteristics of the invention, i.e., they exclude additional unrecited ingredients or steps that detract from novel or basic characteristics of the invention, and they exclude ingredients or steps of the prior art, such as documents in the art that are cited herein or are incorporated by reference herein, especially as it is a goal of this document to define embodiments that are patentable, e.g., novel, nonobvious, inventive, over the prior art, e.g., over documents cited herein or incorporated by reference herein. And, the terms “consists of” and “consisting of” have the meaning ascribed to them in U.S. Patent law; namely, that these terms are closed ended.

### FIELD OF THE INVENTION

The present invention relates to the field of therapeutic immunotherapy, and in particular to the field of active immunotherapy targeted at down-regulating autologous (“self”) proteins and other weakly immunogenic antigens. The invention thus provides novel and improved immunogenic variants of multimeric proteins as well as the necessary tools for the preparation of such variants. The invention further relates to methods of immunotherapy as well as compositions useful in such methods.

### BACKGROUND OF THE INVENTION

Use of active immunotherapy (“vaccination”) as a means of curing or alleviating disease has received growing attention over the last 2 decades. Notably, the use of active immunotherapy as a means for breaking tolerance to autologous proteins that are somehow related to a pathological (or otherwise undesired) physiologic condition has been known since the late seventies where the first experiments with antifertility vaccines where reported.

Vaccines against autologous antigens have traditionally been prepared by “immunogenizing” the relevant self-protein, e.g. by chemical coupling (“conjugation”) to a large foreign and immunogenic carrier protein (cf. U.S. Pat. No. 4,161,519) or by preparation of fusion constructs between the autologous protein and the foreign carrier protein (cf. WO 86/07383). In such constructs, the carrier part of the immunogenic molecule is responsible for the provision epitopes for T-helper lymphocytes (“T_{H} epitopes”) that render possible the breaking of autotolerance.

Later research has proven that although such strategies may indeed provide for the breaking of tolerance against autologous proteins, a number of problems are encountered. Most important is the fact that the immune response that is induced over time will be dominated by the antibodies directed against the carrier portion of the immunogen whereas the reactivity against the autologous protein often declines, an effect that is particularly pronounced when the carrier has previously served as an immunogen—this phenomenon is known as carrier suppression (cf. e.g. Kaliyaperumal et al. 1995., Eur. J. Immunol 25, 3375-3380). However, when using therapeutic vaccination it is usually necessary to re-immunize several times per year and to maintain this treatment for a number of years and this also results in a situation where the immune response against the carrier portion will be increasingly dominant on the expense of the immune response against the autologous molecule.

Further problems involved when using hapten-carrier technology for breaking autotolerance is the negative steric effects exerted by carrier on the autologous protein part in such constructs: The number of accessible B-cell epitopes that resemble the conformational patterns seen in the native autologous protein is often reduced due to simple shielding or masking of epitopes or due to conformational changes induced in the self-part of the immunogen. Finally, it is very often difficult to characterize a hapten-carrier molecule in sufficient detail.

WO 95/05849 provided for a refinement of the above-mentioned hapten-carrier strategies. It was demonstrated that self-proteins wherein is in-substituted as little as one single foreign T_{H} epitope are capable of breaking tolerance towards the autologous protein. Focus was put on the preservation of tertiary structure of the autologous protein in order to ensure that a maximum number of autologous B-cell epitopes would be preserved in the immunogen in spite of the introduction of the foreign T_{H} element. This strategy has generally proven extremely successful inasmuch as the antibodies induced are broad-spectred as well as of high affinity and that the immune response has an earlier onset and a higher titer than that seen when immunizing with a traditional carrier construct.

WO 00/20027 provided for an expansion of the above principle. It was found that introduction of single T_{H} epitopes in the coding sequence for self-proteins could induce cytotoxic T-lymphocytes (CTLs) that reacts specifically with cells expressing the self-protein. The technology of WO 00/20027 also provided for combined therapy, where both antibodies and CTLs are induced-in these embodiments, the immunogens would still be required to preserve a substantial fraction of B-cell epitopes.

WO 95/05849 and WO 98/46642 both disclose vaccine technology that is suitable for down-regulating the activity of TNFα (tumour necrosis factor α), a cytokine involved in the pathology of several diseases such as type I diabetes, rheumatoid arthritis, and inflammatory bowel disease. Both disclosures teach preservation of the tertiary structure of monomer TNFα when this molecule confronts the immune system.

WO 00/65058 relates to down-regulation of interleukin 5 (IL5), a molecule involved in the activation of eosinophil granulocyte activity that is important in the pathogenesis of a number of airway diseases such as chronic asthma. It is taught that down-regulation can be accomplished by means of both polypeptide vaccination technology, live vaccines and nucleic acid vaccination and it is further taught that the preservation of B-cell epitopes is important if raising an immune response against IL5.

Even though the above-referenced technologies have provided for very promising results, there are several factors that may come into play when assessing the viability of a vaccine approach in combating a disease. One of these factors is the expression level of the immunogenic protein.

For instance, in order for a nucleic acid vaccine to be functional, the cells transfected in vivo with a construct encoding an “immunogenized” autologous protein must be able to express the immunogen in sufficient amounts so as to induce a suitable immune response. Also, polypeptide based vaccines require that the immunogenic protein can be produced in satisfactory amounts in an industrial fermentation process. However, it is often observed that even slight changes in the amino acid sequence of a known protein can have dramatic effects on the amounts of protein that can be recovered.

Further, the stability of genetically modified protein sequences may also be less than optimal (both in terms of shelf-life and in terms of stability in vivo).

Finally, when the self-protein that it is desired to down-regulate is a heteropolymer or homopolymer it is not necessarily so that a variant of a monomeric unit of this protein will be capable of inducing antibodies that are sufficiently specific for the conformation native to the polymeric protein.

### OBJECT OF THE INVENTION

It is an object of the invention to provide for improved immunogenic analogues of polymeric autologous proteins as well as to provide for improved methods for inducing humoral immunity against such polymeric autologous proteins. It is a further object to provide for immunogenic analogues of self-proteins that have an improved stability and exhibit improved characteristics when expressed in heterologous host cells. Finally, it is also objects of the invention to provide for means and measures that are useful when preparing or utilising the improved immunogens.

### SUMMARY OF THE INVENTION

When producing large-scale amounts of recombinant protein in bacterial host cells, it is often desired that the expression product becomes available as inclusion bodies inside the bacteria. The reasons for this are sevarel: For example the expression yields are normally considerably higher when the protein is expressed as insoluble inclusion bodies, and the purification of the protein is also facilitated because the desired expression product is easily and conveniently separated from soluble protein from the bacterial fermentation.

When expressing a recombinant protein as insoluble inclusion bodies, it is often necessary to subject the expression product to various protein refolding processes in order to obtain it in a biologically active form, but this is normally acceptable even though such a step leads to a certain loss of total recombinant protein that is never folded into the correct biologically active form.

However, when producing recombinant immunogenic variants of non-immunogenic self-proteins it is necessary to introduce T_{H} epitopes and thereby the primary structure of the protein product becomes altered when compared to the native self-protein. The present inventors have experienced that even the slightest of changes renders the traditional approach of inclusion body expression followed by refolding impractical: The yields of protein after refolding that has preserved a satisfactory fraction of B-cell epitopes compared to the native self-protein are very often low, and this problem increases with the complexity of the protein in question.

It has now been found that designing and effecting expression of protein constructs that are produced as soluble protein from bacteria is a superior way of preparing immunogenic variants of self-proteins—even though subsequent purification steps become more complicated because other soluble proteins have to be removed, the final purified and correctly folded product is obtained in significantly higher yields than when compared to the traditional approach outlined above. And, very importantly, the purified proteins obtained from this type of expression exhibit a hitherto unprecedented ability to preserve B-cell epitopes of the native self-protein from which they are derived.

In brief, according to the present invention, soluble expression of variant proteins is an excellent selection criterion when initially selecting for immunogenic variants of a self-protein that are suitable for vaccination purposes.

In order to obtain the goal of soluble protein expression of such immunogenized self-proteins (and other proteins where changes have been introduced in the primary sequence), a number of parameters can be varied—multimeric proteins that are difficult to assemble can be produce by stabilising their structure both on the monomeric level but also by preparing monomeric mimicks of the multimer, and also simple monomeric proteins can be stabilised according to the teachings set forth herein.

Another important factor is the fermentation conditions—findings in the present inventors' lab have e.g. indicated that fermentation of bacteria at lower temperatures than those normally used for obtaining high level expression greatly facilitate the production of soluble forms of the variant proteins.

The present inventors have found that preparation of “monomerized” forms of IL5 and TNFα may provide for immunogenic molecules having a high stability, superior immunogenicity and desirable production characteristics. In particular, the yield of protein is surprisingly high when expressing recombinant polypeptides constituted by two monomers of hIL5 joined by means of a peptide linker and including foreign T helper cell epitopes. It is believed that this finding constitutes a general applicable finding relating to multimeric proteins, the quarternary structure of which allows for tailoring of a monomeric version thereof.

It is believed that the present technology is especially suited for preparing immunogens for breaking autotolerance against autologous proteins, since the introduction of the peptide linker can be elegantly combined with the provision of foreign T helper epitopes while at the same time preserving the 3D structure of the multimeric protein (i.e. preservation of both elements from tertiary and from quarternary structure of such a protein, by imposing the original quarternary structure on the new tertiary structure in the monomeric protein).

Hence, in one broad aspect, the invention relates to an immunogenic analogue of a polymeric protein, said polymeric protein consisting (in nature) of at least 2 monomeric units that are not joined by means of a peptide bond, wherein said analogue

a) includes substantial fragments of at least 2 monomeric units of said polymeric protein, wherein said substantial fragments are joined via peptide bonds through a peptide linker,

b) includes at least one MHC Class II binding amino acid sequence that is heterologous to the polymeric protein, and

c) can be produced as one single expression product from a cell harbouring an expression vector encoding the analogue.

The present inventors have also found that a number of particular manipulations in the amino acid sequence of monomeric TNFα results in the provision of monomer molecules that are both immunogenic and capable of attaining a functional quarternary structure, meaning that these molecules has so high degree of preserved tertiary structure that they spontaneously can form functional, receptor binding, dimers and trimers, and also that these monomers are produced as soluble proteins in bacteria.

Some of these manipulations that have been performed in the TNFα protein are believed to be generally applicable for proteins where it is desired to prepare a stabilised tertiary structure compared to a native protein.

A particular aspect of the invention relates to a number of variations in the TNFα monomer structure that are sufficiently non-destructive so as to allow correct folding of the TNFα monomers while at the same time introducing at least one MHC Class II binding amino acid sequence. It has e.g. been found that insertion of a foreign T_{H} epitope can be made in one particular loop structure in native TNFα without this having a negative impact on the expression characteristics of the protein or on the monomer's capability of forming a functional TNFα dimer or triimer. Hence, a important part of the invention relates to an immunogenic analogue of human TNFα, wherein the analogue includes at least one foreign MHC Class II binding amino acid sequence and further has the characteristic of being

a human TNFα monomer or a monomerized analogue of TNFα of the present invention, wherein has been inserted or in-substituted at least one foreign MHC Class II binding amino acid sequence into flexible loop 3, and/or

a human TNFα monomer or a monomerized analogue of TNFα of the present invention, wherein has been introduced at least one disulfide bridge that stabilises the TNFα monomer 3D structure, and/or

a human TNFα monomer or a monomerized analogue of TNFα of the present invention, wherein any one of amino acids 1, 2, 3, 4, 5, 6, 7, 8, and 9 in the amino terminus have been deleted, and/or

a human TNFα monomer or a monomerized analogue of TNFα of the present invention, wherein an inserted or in-substituted at least one foreign MHC Class II binding amino acid sequence into loop 1 in an intron position, and/or

a human TNFα monomer or a monomerized analogue of TNFα of the present invention, wherein at least one foreign MHC Class II binding amino acid sequence is introduced as part of an artificial stalk region in the N-terminus of human TNFα, and/or

a human TNFα monomer or a monomerized analogue of TNFα of the present invention, wherein at least one foreign MHC Class II binding amino acid sequence is introduced so as to stabilize the monomer structure by increasing the hydrophobicity of the trimeric interaction interface, and/or

a human TNFα monomer or a monomerized analogue of TNFα of the present invention, wherein at least one foreign MHC Class II binding amino acid sequence flanked by glycine residues is inserted or in-substituted in the TNFα amino acid sequence, and/or

a human TNFα monomer or a monomerized analogue of TNFα of the present invention, wherein at least one foreign MHC Class II binding amino acid sequence is inserted or in-substituted in the D-E loop, and/or

a human TNFα monomer or a monomerized analogue of TNFα of the present invention, wherein at least one foreign MHC Class II binding amino acid sequence is inserted or in-substituted between two identical subsequences of human TNFα, and/or

a human TNFα monomer or a monomerized analogue of TNFα of the present invention, wherein at least one salt bridge in human TNFα has been strengthened or substituted with a disulphide bridge, and/or

a human TNFα monomer or a monomerized analogue of TNFα of the present invention, wherein solubility and/or stability towards proteolysis is enhanced by introducing mutations that mimic murine TNFα crystalline structure, and/or

a human TNFα monomer or a monomerized analogue of TNFα of the present invention, wherein potential toxicity is reduced or abolished by introduction of at least one point mutation.

In general, it has been found that all of the best suited immunogenic analogues of the invention are those that are soluble proteins already at the stage when they are produced and isolated in soluble form from their recombinant host cells.

The invention further provides for nucleic acid fragments (such as DNA fragments) encoding such immunogenic analogues and also to vectors including such DNA fragments.

The invention also provides for transformed cells useful for preparing the analogues.

The invention further provides for immunogenic compositions comprising the analogous or the vectors of the invention.

Also provided by the invention are methods of treatment, where multimeric proteins are down-regulated and to treatment of speicific diseases related to the particular multimeric proteins.

### LEGEND TO THE FIGURE

FIG. 1: The p2ZOP2f insect cell expression vector. The sequence of the vector is set forth in SEQ ID NO: 60. The vector contains a multi-cloning site (MCS) downstream the OpIE2 promoter and upstream of an OpIE2 poly A tail (OpIE2pA). The marker zeocin resistance gene (ZeoR) is under the control of a second OpIE2 promoter.

### DETAILED DISCLOSURE OF THE INVENTION

Definitions

In the following, a number of terms used in the present specification and claims will be defined and explained in detail in order to clarify the metes and bounds of the invention.

The terms “T-lymphocyte” and “T-cell” will be used interchangeably for lymphocytes of thymic origin that are responsible for various cell mediated immune responses as well as for helper activity in the humeral immune response. Likewise, the terms “B-lymphocyte” and “B-cell” will be used interchangeably for antibody-producing lymphocytes.

A “polymeric protein” is herein defined as a protein that includes at least two polypeptide chains that are not joined end-to-end via a peptide bond (the term “multimeric protein” is used interchangeably therewith). Hence, polymeric proteins may be polymers consisting of several polypeptides that are kept together in polymeric form by means of disulfide bonds and/or non-covalent binding. Also included within the term are processed pre-proteins and pro-proteins that after processing include at least two free C-termini and at least two free N-termini. Finally, included within the term is also temporarily existing complexes between at least two polypeptides that may form up an unstable but yet biologically active molecular entity that has a distinct 3-dimensional structure.

“An immunogenic analogue” (or an “immunogenized” analogue or variant) is herein meant to designate a single polypeptide that includes substantial parts of the sequence information found in a complete polymeric protein. That is, the analogue protein of the invention includes one polypeptide chain whereas a polymeric protein includes at least 2 polypeptide chains. It should be noted that the analogue may be a variation of the polymers monomeric subunit structure, but in that case, the immunogenic analogue is capable of forming polymeric protein complexes that resemble the native polymer.

A “monomerized” analogue or variant of a polymeric protein is in the present context a single polypeptide that includes, in covalently linked form via a peptide bond, at least 2 polypeptide chains found in a polymeric protein in nature, where these 2 polypeptide chains are not linked via a peptide bond.

“A substantial fragment” of a monomeric unit of a multimeric protein is intended to mean a part of a monomeric polypeptide that constitutes at least enough of the monomeric polypeptide so as to form a domain that folds up in substantially the same 3D conformation as can be found in the multimeric protein.

An “IL5 polypeptide” is herein intended to denote polypeptides having the amino acid sequence of IL5 proteins derived from humans and other mammals. Also unglycosylated forms of IL5 which are prepared in prokaryotic system are included within the boundaries of the term as are forms having varying glycosylation patterns due to the use of e.g. yeasts or other non-mammalian eukaryotic expression systems. It should, however, be noted that when using the term “an IL5 polypeptide” it is intended that the polypeptide in question is normally non-immunogenic when presented to the animal to be treated. In other words, the IL5 polypeptide is a self-protein or is a xeno-analogue of such a self-protein which will not normally give rise to an immune response against IL5 of the animal in question.

A “TNFα polypeptide” is herein intended to denote polypeptides having the amino acid sequence of TNFα proteins derived from humans and other mammals. Also unglycosylated forms of TNFα which are prepared in prokaryotic system are included within the boundaries of the term as are forms having varying glycosylation patterns due to the use of e.g. yeasts or other non-mammalian eukaryotic expression systems. It should, however, be noted that when using the term “a TNFα polypeptide” it is intended that the polypeptide in question is normally non-immunogenic when presented to the animal to be treated. In other words, the TNFα polypeptide is a self-protein or is a xeno-analogue of such a self-protein which will not normally give rise to an immune response against TNFα of the animal in question.

An “IL5 analogue” is an IL5 polypeptide which has been either subjected to changes in its primary structure and/or that is associated with elements from other molecular species. Such a change can e.g. be in the form of fusion of an IL5 polypeptide to a suitable fusion partner (i.e. a change in primary structure exclusively involving C- and/or N-terminal additions of amino acid residues) and/or it can be in the form of insertions and/or deletions and/or substitutions in the IL5 polypeptide's amino acid sequence. Also encompassed by the term are derivatized IL5 molecules, cf. the discussion below of modifications of IL5.

A “TNFα analogue” is a TNFα polypeptide which has been either subjected to changes in its primary structure and/or that is associated with elements from other molecular species. Such a change can e.g. be in the form of fusion of a TNFα polypeptide to a suitable fusion partner (i.e. a change in primary structure exclusively involving C- and/or N-terminal additions of amino acid residues) and/or it can be in the form of insertions and/or deletions and/or substitutions in the TNFα polypeptide's amino acid sequence. Also encompassed by the term are derivatized TNFα molecules, cf. the discussion below of modifications of TNFα.

It will be understood, that IL5 and TNFα analogues also include monomeric variants that contains substantial parts of complete IL5 and TNFα multimeric proteins.

When using the abbreviations “IL5” and “TNFα” herein, this is intended as references to the amino acid sequences of mature, wildtype IL5 and TNFα (also denoted “IL5m” and “IL5wt” as well as “TNFαm” and “TNFαwt” herein), respectively. Mature human IL5 is denoted hIL5, hIL5m or hIL5wt, and murine mature IL5 is denoted mIL5, mIL5m, or mIL5wt and a similar syntax is used for TNFα. In cases where a DNA construct includes information encoding a leader sequence or other material, this will normally be clear from the context.

The term “polypeptide” is in the present context intended to mean both short peptides of from 2 to 10 amino acid residues, oligopeptides of from 11 to 100 amino acid residues, and polypeptides of more than 100 amino acid residues. Furthermore, the term is also intended to include proteins, i.e. functional biomolecules comprising at least one polypeptide; when comprising at least two polypeptides, these may form complexes, be covalently linked, or may be non-covalently linked. The polypeptide(s) in a protein can be glycosylated and/or lipidated and/or comprise prosthetic groups.

The term “subsequence” means any consecutive stretch of at least 3 amino acids or, when relevant, of at least 3 nucleotides, derived directly from a naturally occurring IL5 amino acid sequence or nucleic acid sequence, respectively.

The term “animal” is in the present context in general intended to denote an animal species (preferably mammalian), such as Homo sapiens, Canis domesticus, etc. and not just one single animal. However, the term also denotes a population of such an animal species, since it is important that the individuals immunized according to the method of the invention all harbour substantially the same ILS allowing for immunization of the animals with the same immunogen(s). If, for instance, genetic variants of IL5 of TNFα exist in different human populations it may be necessary to use different immunogens in these different populations in order to be able to break the autotolerance towards IL5 and TNFα, respectively, in each population. It will be clear to the skilled person that an animal in the present context is a living being which has an immune system. It is preferred that the animal is a vertebrate, such as a mammal.

By the term “down-regulation” is herein meant reduction in the living organism of the biological activity of the multimeric protein (e.g. by interference with the interaction between the multimeric protein and biologically important binding partners for this molecule). The down-regulation can be obtained by means of several mechanisms: Of these, simple interference with the active site in the multimeric protein by antibody binding is the most simple. However, it is also within the scope of the present invention that the antibody binding results in removal of the multimeric protein by scavenger cells (such as macrophages and other phagocytic cells).

The expression “effecting presentation . . . to the immune system” is intended to denote that the animal's immune system is subjected to an immunogenic challenge in a controlled manner. As will appear from the disclosure below, such challenge of the immune system can be effected in a number of ways of which the most important are vaccination with polypeptide containing “pharmaccines” (i.e. a vaccine which is administered to treat or ameliorate ongoing disease) or nucleic acid “pharmaccine” vaccination. The important result to achieve is that immune competent cells in the animal are confronted with the antigen in an immunologically effective manner, whereas the precise mode of achieving this result is of less importance to the inventive idea underlying the present invention.

The term “immunogenically effective amount” has its usual meaning in the art, i.e. an amount of an immunogen which is capable of inducing an immune response which significantly engages pathogenic agents which share immunological features with the immunogen.

When using the expression that the IL5, TNFα or other self-protein has been “modified” is herein meant a chemical modification of the polypeptide which constitutes the backbone of the self-protein. Such a modification can e.g. be derivatization (e.g. alkylation, acylation, esterification etc.) of certain amino acid residues in the amino acid sequence, but as will be appreciated from the disclosure below, the preferred modifications comprise changes of (or additions to) the primary structure of the amino acid sequence.

When discussing “autotolerance towards an autologous protein” it is understood that since the relevant multimeric protein is a self-protein in the population to be vaccinated, normal individuals in the population do not mount an immune response against it; it cannot be excluded, though, that occasional individuals in an animal population might be able to produce antibodies against the native multimer, e.g. as part of an autoimmune disorder. At any rate, an animal species will normally only be autotolerant towards its own multimer, but it cannot be excluded that analogues derived from other animal species or from a population having a different phenotype would also be tolerated by said animal.

A “foreign T-cell epitope” (or: “foreign T-lymphocyte epitope”) is a peptide which is able to bind to an MHC molecule and which stimulates T-cells in an animal species—an alternate term is therefore. Preferred foreign T-cell epitopes in the invention are “promiscuous” (or “universal” or “broad-range”) epitopes, i.e. epitopes that bind to a substantial fraction of a particular class of MHC molecules in an animal species or population. Only a very limited number of such promiscuous T-cell epitopes are known, and they will be discussed in detail below. It should be noted that in order for the immunogens which are used according to the present invention to be effective in as large a fraction of an animal population as possible, it may be necessary to 1) insert several foreign T-cell epitopes in the same analogue or 2) prepare several analogues wherein each analogue has a different promiscuous epitope inserted. It should be noted also that the concept of foreign T-cell epitopes also encompasses use of cryptic T-cell epitopes, i.e. epitopes which are derived from a self-protein and which only exerts immunogenic behaviour when existing in isolated form without being part of the self-protein in question.

A “foreign T helper lymphocyte epitope” (a foreign T_{H} epitope) is a foreign T cell epitope which binds an MHC Class II molecule and can be presented on the surface of an antigen presenting cell (APC) bound to the MHC Class II molecule.

An “MHC Class II binding amino acid sequence that is heterologous to a multimeric protein” is therefore an MHC Class II binding peptide that does not exist in the multimeric protein in question. Such a peptide will, if it is also truly foreign to the animal species harbouring the multimeric protein, be a foreign T_{H} epitope.

A “functional part” of a (bio)molecule is in the present context intended to mean the part of the molecule which is responsible for at least one of the biochemical or physiological effects exerted by the molecule. It is well-known in the art that many enzymes and other effector molecules have an active site which is responsible for the effects exerted by the molecule in question. Other parts of the molecule may serve a stabilizing or solubility enhancing purpose and can therefore be left out if these purposes are not of relevance in the context of a certain embodiment of the present invention. However, according to the present invention, it is preferred to utilise as much of the polymeric molecule as possible, because the increased stability has in fact been demonstrated when using the monomers described herein.

The term “adjuvant” has its usual meaning in the art of vaccine technology, i.e. a substance or a composition of matter which is 1) not in itself capable of mounting a specific immune response against the immunogen of the vaccine, but which is 2) nevertheless capable of enhancing the immune response against the immunogen. Or, in other words, vaccination with the adjuvant alone does not provide an immune response against the immunogen, vaccination with the immunogen may or may not give rise to an immune response against the immunogen, but the combination of vaccination with immunogen and adjuvant induces an immune response against the immunogen which is stronger than that induced by the immunogen alone.

“Targeting” of a molecule is in the present context intended to denote the situation where a molecule upon introduction in the animal will appear preferentially in certain tissue(s) or will be preferentially associated with certain cells or cell types. The effect can be accomplished in a number of ways including formulation of the molecule in composition facilitating targeting or by introduction in the molecule of groups which facilitates targeting. These issues will be discussed in detail below.

“Stimulation of the immune system” means that a substance or composition of matter exhibits a general, non-specific immunostimulatory effect. A number of adjuvants and putative adjuvants (such as certain cytokines) share the ability to stimulate the immune system. The result of using an immunostimulating agent is an increased “alertness” of the immune system meaning that simultaneous or subsequent immunization with an immunogen induces a significantly more effective immune response compared to isolated use of the immunogen.

Characteristics of the Immunogenic Analogues of the Invention

The polymeric proteins that are the targets of the presently disclosed strategies may be both homopolymers and heteropolymers. As will be clear from the examples, the most important feature in the first aspect of the invention is that the polymeric protein in question can be “monomerized” without introducing significant changes in the 3 dimensional structure of the multimeric protein. Hence, the particular function of the multimeric protein is not important for the gist of the present invention—rather it is the structural characteristics of the protein that decides whether or not it is a suitable candidate for the present approach in the first aspect of the invention. For instance, if the N-terminus of one monomer in the multimeric protein has a spatial proximity to the C-terminus of another monomer in the multimer, the linking of these two particular monomers via a peptide linker may be accomplished without imposing significant changes relative to the structure of the native multimeric protein. If, on the other hand, the termini are far apart, the practice of the present invention requires that large parts of at least one of the monomers is irrelevant for the immunogenic purpose of the invention or that linking between monomeric subunits can be done with a long linker peptide without this having a negative impact on the antigenic characteristics of the protein.

In the second aspect of the invention, the “immunogenization” of the self-protein monomer unit is made in such a way, that the resulting variant monomer is still capable of forming part of a polymer protein that shares the quarternary structure of the native polymeric self-protein.

It is advantageous if the immunogenic analogue according to the invention displays, in the substantial fragments, a substantial fraction of B-cell epitopes found in the corresponding monomers when being part of the polymeric protein. A substantial fraction of B-cell epitopes is herein intended to mean a fraction of B-cell epitopes that antigenically characterises the multimeric protein versus other proteins. It is preferred that the substantial fragments display essentially all B-cell epitopes found in the corresponding monomers when being part of the polymeric protein—of course, introduction of minor changes in the monomer sequence may be necessary. For instance an amino acid sequence derived from a monomeric unit may be modified by means of amino acid insertion, substitution, deletion or addition so as to reduce toxicity of the analogue as compared to the multimeric protein and/or so as to introduce the MHC Class II binding amino acid sequence, if it is undesired to have that sequence positioned in a linker.

An especially preferred embodiment provides for an immunogenic analogue of the invention, wherein each of the substantial fractions comprises essentially the complete amino acid sequence of each monomeric unit, either as a continuous sequence or as a sequence including inserts. That is, only insignificant parts of the monomeric unit's sequence are left out of the analogue, e.g. in cases where such a sequence does not contribute to tertiary structure of the monomeric unit or quarternary structure of the multimeric protein. However, this embodiment allows for substitution or insertion of the monomer, as long as the 3D structure of the multimeric protein is maintained. Hence, it is especially advantageous if the immunogenic analogue is one, wherein amino acid sequences of all monomeric units of the polymeric proteins are represented in the analogue, and it is particularly advantageous if the analogue includes the complete amino acid sequences of (all) the monomers constituting the polymeric protein, either as unbroken sequences or as sequences including inserts.

As will appear, it is therefore preferred that the 3-dimensional structure of the complete polymeric protein is essentially preserved in the analogue.

Demonstration of maintenance of a substantial fraction of B-cell epitopes or even the 3-dimensional structure of a multimeric protein that is subjected to modification as described herein can be achieved in several ways. One is simply to prepare a polyclonal antiserum directed against the multimer (e.g. an antiserum prepared in a rabbit) and thereafter use this antiserum as a test reagent (e.g. in a competitive ELISA) against the modified proteins which are produced. Modified versions (analogues) which react to the same extent with the antiserum as does the multimer must be regarded as having the same 3D structure as the multimer whereas analogues exhibiting a limited (but still significant and specific) reactivity with such an antiserum are regarded as having maintained a substantial fraction of the original B-cell epitopes.

Alternatively, a selection of monoclonal antibodies reactive with distinct epitopes on the multimer can be prepared and used as a test panel. This approach has the advantage of allowing 1) an epitope mapping of the multimer and 2) a mapping of the epitopes which are maintained in the analogues prepared.

Of course, a third approach would be to resolve the 3-dimensional structure of the multimer (cf. above) and compare this to the resolved three-dimensional structure of the analogues prepared. Three-dimensional structure can be resolved by the aid of X-ray diffraction studies and NMR-spectroscopy. Further information relating to the tertiary structure can to some extent be obtained from circular dichroism studies which have the advantage of merely requiring the polypeptide in pure form (whereas X-ray diffraction requires the provision of crystallized polypeptide and NMR requires the provision of isotopic variants of the polypeptide) in order to provide useful information about the tertiary structure of a given molecule. However, ultimately X-ray diffraction and/or NMR are necessary to obtain conclusive data since circular dichroism can only provide indirect evidence of correct 3-dimensional structure via information of secondary structure elements.

The immunogenic analogue of the invention may include a peptide linker that includes or contributes to the presence in the analogue of at least one MHC Class II binding amino acid sequence that is heterologous to the multimeric protein. This is particularly useful in those cases where it is undesired to alter the amino acid sequence corresponding to monomeric units in the multimeric protein. Alternatively, the peptide linker may be free of and not contributing to the presence of an MHC Class II binding amino acid sequence in the animal species from where the multimeric protein is derived; this can conveniently be done in cases where it is necessary to utilise a very short linker or where it is advantageous to e.g. detoxify a potentially toxic analogue by introducing the MHC Class II binding element in an active site. Both these embodiments can be combined with introduction of point mutations that detoxify the molecule if need be.

It is preferred that the MHC Class II binding amino acid sequence binds a majority of MHC Class II molecules from the animal species from where the multimeric protein has been derived, i.e. that the MHC Class II binding amino acid sequence is universal or promiscuos.

It is of course important that this sequence serves its purpose as a T cell epitope in the species for which the immunogen is intended to serve as a vaccine constituent. There exists a number of naturally occurring “promiscuous” T-cell epitopes which are active in a large proportion of individuals of an animal species or an animal population and these are preferably introduced in the vaccine, thereby reducing the need for a very large number of different analogues in the same vaccine. Hence, the at least one MHC Class II binding amino acid sequence is preferably selected from a natural T-cell epitope and an artificial MHC-II binding peptide sequence. Especially preferred sequences are a natural T-cell epitope is selected from a Tetanus toxoid epitope such as P2 (SEQ ID NO: 3) or P30 (SEQ ID NO: 5), a diphtheria toxoid epitope, an influenza virus hemagluttinin epitope, and a P. falciparum CS epitope.

Over the years a number of other promiscuous T-cell epitopes have been identified. Especially peptides capable of binding a large proportion of HLA-DR molecules encoded by the different HLA-DR alleles have been identified and these are all possible T-cell epitopes to be introduced in the analogues used according to the present invention. Cf. also the epitopes discussed in the following references which are hereby all incorporated by reference herein: WO 98/23635 (Frazer I H et al., assigned to The University of Queensland); Southwood S et. al, 1998, J. Immunol. 160: 3363-3373; Sinigaglia F et al., 1988, Nature 336: 778-780; Chicz RM et al., 1993, J. Exp. Med 178: 27-47; Hammer J et al., 1993, Cell 74: 197-203; and Falk K et al., 1994, Immunogenetics 39: 230-242. The latter reference also deals with HLA-DQ and -DP ligands. All epitopes listed in these 5 references are relevant as candidate natural epitopes to be used in the present invention, as are epitopes that share common motifs with these.

Alternatively, the epitope can be any artificial T-cell epitope which is capable of binding a large proportion of MHC Class II molecules. In this context the pan DR epitope peptides (“PADRE”) described in WO 95/07707 and in the corresponding paper Alexander J et al., 1994, Immunity 1: 751-761 (both disclosures are incorporated by reference herein) are interesting candidates for epitopes to be used according to the present invention. It should be noted that the most effective PADRE peptides disclosed in these papers carry D-amino acids in the C- and N-termini in order to improve stability when administered. However, the present invention primarily aims at incorporating the relevant epitopes as part of the analogue which should then subsequently be broken down enzymatically inside the lysosomal compartment of APCs to allow subsequent presentation in the context of an MHC-II molecule and therefore it is not expedient to incorporate D-amino acids in the epitopes used in the present invention.

One especially preferred PADRE peptide is the one having the amino acid sequence AKFVAAWTLKAAA (SEQ ID NO: 7) or an immunologically effective subsequence thereof. This, and other epitopes having the same lack of MHC restriction are preferred T-cell epitopes which should be present in the analogues used in the inventive method. Such super-promiscuous epitopes will allow for the most simple embodiments of the invention wherein only one single modified IL5 is presented to the vaccinated animal's immune system.

Preferred embodiments of the invention includes modification by introducing at least one foreign immunodominant T_{H} epitope. It will be understood that the question of immune dominance of a T_{H} epitope depends on the animal species in question. As used herein, the term “immunodominance” simply refers to epitopes which in the vaccinated individual gives rise to a significant immune response, but it is a well-known fact that a T_{H} epitope which is immunodominant in one individual is not necessarily immunodominant in another individual of the same species, even though it may be capable of binding MHC-II molecules in the latter individual.

As mentioned above, the introduction of a foreign T-cell epitope can be accomplished by introduction of at least one amino acid insertion, addition, deletion, or substitution. Of course, the normal situation will be the introduction of more than one change in the amino acid sequence (e.g. insertion of or substitution by a complete T-cell epitope) but the important goal to reach is that the analogue, when processed by an antigen presenting cell (APC), will give rise to such a T-cell epitope being presented in context of an MCH Class II molecule on the surface of the APC. Thus, if the amino acid sequence of the monomeric unit in appropriate positions comprises a number of amino acid residues which can also be found in a foreign T_{H} epitope then the introduction of a foreign T_{H} epitope can be accomplished by providing the remaining amino acids of the foreign epitope by means of amino acid insertion, addition, deletion and substitution. In other words, it is not necessary to introduce a complete T_{H} epitope by insertion or substitution.

According to the present invention, the analogue may also form part of larger molecule wherein it is coupled to at least one functional moiety, the presence of which does not interfer negatively to a significant degree with the antibody-accessability of the analogue. The nature of such moieties (which may be fused to the analogue) can be to target the modified molecule to an antigen presenting cell (APC) or a B-lymphocyte, to stimulate the immune system, and to optimize presentation of the analogue to the immune system.

Targeting moieties are conveniently selected from the group consisting of a substantially specific binding partner for a B-lymphocyte specific surface antigen or for an APC specific surface antigen, such as a hapten or a carbohydrate for which there is a receptor on the B-lymphocyte or the APC. The immunestimulating moieties may be selected from the group consisting of a cytokine, a hormone, and a heat-shock protein. The presentation optimising moiety may be selected from the group consisting of a lipid group, such as a palmitoyl group, a myristyl group, a farnesyl group, a geranyl-geranyl group, a GPI-anchor, and an N-acyl diglyceride group.

A suitable cytokine is, or is an effective part of any of, interferon γ (IFN-g), Flt3L, interleukin 1 (IL-1), interleukin 2 (IL-2), interleukin 4 (IL-4), interleukin 6 (IL-6), interleukin 12 (IL-12), interleukin 13 (IL-13), interleukin 15 (IL-15), and granulocyte-macrophage colony stimulating factor (GM-CSF).

A preferred heat-shock protein is, or is an effective part of any of, HSP70, HSP90, HSC70, GRP94, and calreticulin (CRT).

It is preferred that the number of amino acid insertions, deletions, substitutions or additions is at least 2, such as 3, 4, 5, 6, 7, 8, 9, 10, 11, 12, 13, 14, 15, 16, 17, 18, 19, 20, 21, and 25 insertions, substitutions, additions or deletions. It is furthermore preferred that the number of amino acid insertions, substitutions, additions or deletions is not in excess of 150, such as at most 100, at most 90, at most 80, and at most 70. It is especially preferred that the number of substitutions, insertions, deletions, or additions does not exceed 60, and in particular the number should not exceed 50 or even 40. Most preferred is a number of not more than 30. With respect to amino acid additions, it should be noted that these, when the resulting construct is in the form of a fusion polypeptide, is often considerably higher than 150.

Preferred embodiments of the invention includes modification by introducing at least one foreign immunodominant T_{H} epitope (=“foreign MHC Class II binding amino acid sequence”). It will be understood that the question of immune dominance of a T_{H} epitope depends on the animal species in question. As used herein, the term “immunodominance” simply refers to epitopes which in the vaccinated individual gives rise to a significant immune response, but it is a well-known fact that a T_{H} epitope which is immunodominant in one individual is not necessarily immunodominant in another individual of the same species, even though it may be capable of binding MHC-II molecules in the latter individual.

Another important point is the issue of MHC restriction of T_{H} epitopes. In general, naturally occurring T_{H} epitopes are MHC restricted, i.e. a certain peptide constituting a T_{H} epitope will only bind effectively to a subset of MHC Class II molecules. This in turn has the effect that in most cases the use of one specific T_{H} epitope will result in a vaccine component which is effective in a fraction of the population only, and depending on the size of that fraction, it can be necessary to include more T_{H} epitopes in the same molecule, or alternatively prepare a multi-component vaccine wherein the components are variants which are distinguished from each other by the nature of the T_{H} epitope introduced.

If the MHC restriction of the T-cells used is completely unknown (for instance in a situation where the vaccinated animal has a poorly defined MHC composition), the fraction of the animal population covered by a specific vaccine composition can be determined by means of the following formula:

$${f}_{population} = 1 - {\prod }_{i = 1}^{n} ( 1 - {p}_{i} ) \qquad ( II )$$

where pᵢ is the frequency in the population of responders to the i^{th} foreign T-cell epitope present in the vaccine composition, and n is the total number of foreign T-cell epitopes in the vaccine composition. Thus, a vaccine composition containing 3 foreign T-cell epitopes having response frequencies in the population of 0.8, 0.7, and 0.6, respectively, would give

1−0.2×0.3×0.4=0.976

i.e. 97.6 percent of the population will statistically mount an MHC-II mediated response to the vaccine.

The above formula does not apply in situations where a more or less precise MHC restriction pattern of the peptides used is known. If, for instance a certain peptide only binds the human MHC-II molecules encoded by HLA-DR alleles DR1, DR3, DR5, and DR7, then the use of this peptide together with another peptide which binds the remaining MHC-II molecules encoded by HLA-DR alleles will accomplish 100% coverage in the population in question. Likewise, if the second peptide only binds DR3 and DR5, the addition of this peptide will not increase the coverage at all. If one bases the calculation of population response purely on MHC restriction of T-cell epitopes in the vaccine, the fraction of the population covered by a specific vaccine composition can be determined by means of the following formula:

$${f}_{population} = 1 - {\prod }_{j = 1}^{3} {( 1 - {ϕ}_{i} )}^{2} \qquad ( III )$$

wherein φⱼ is the sum of frequencies in the population of allelic haplotypes encoding MHC molecules which bind any one of the T-cell epitopes in the vaccine and which belong to the j^{th} of the 3 known HLA loci (DP, DR and DQ); in practice, it is first determined which MHC molecules will recognize each T-cell epitope in the vaccine and thereafter these MHC molecules are listed by type (DP, DR and DQ)—then, the individual frequencies of the different listed allelic haplotypes are summed for each type, thereby yielding φ₁, φ₂, and φ₃.

It may occur that the value pᵢ in formula II exceeds the corresponding theoretical value πᵢ:

$${π}_{i} = 1 - {\prod }_{j = 1}^{3} {( 1 - {v}_{j} )}^{2} \qquad ( IV )$$

wherein vⱼ is the sum of frequencies in the population of allelic haplotypes encoding MHC molecules which bind the i^{th} T-cell epitope in the vaccine and which belong to the j^{th} of the 3 known HLA loci (DP, DR and DQ). This means that in 1−πᵢ of the population there is a frequency of responders of f_{residual}_{_{—}}ᵢ₌₍ₚᵢ−πᵢ)/(1−πᵢ). Therefore, formula III can be adjusted so as to yield formula V:

$${f}_{population} = 1 - {\prod }_{j = 1}^{3} {( 1 - {ϕ}_{j} )}^{2} + ( 1 - {\prod }_{i = 1}^{n} ( 1 - {f}_{residual\_i} ) ) \qquad ( V )$$

where the term 1−f_{residual}_{_{—}}ᵢ is set to zero if negative. It should be noted that formula V requires that all epitopes have been haplotype mapped against identical sets of haplotypes.

Therefore, when selecting T-cell epitopes to be introduced in the analogue of the invention, it is important to include all knowledge of the epitopes which is available: 1) The frequency of responders in the population to each epitope, 2) MHC restriction data, and 3) frequency in the population of the relevant haplotypes.

It should be noted that preferred analogues of the invention comprise modifications which results in a polypeptide that includes stretches having a sequence identity of at least 70% with the corresponding monomeric units of the multimermic protein or with subsequences thereof of at least 10 amino acids in length. Higher sequence identities are preferred, e.g. at least 75% or even at least 80% or 85%. The sequence identity for proteins and nucleic acids can be calculated as (N_{ref}−N_{dif})·100/N_{ref}, wherein N_{dif} is the total number of non-identical residues in the two sequences when aligned and wherein N_{ref} is the number of residues in one of the sequences. Hence, the DNA sequence AGTCAGTC will have a sequence identity of 75% with the sequence AATCAATC (N_{dif}=2 and N_{ref}=8).

Finally, in order to conclusively verify that an analogue of the invention is indeed effective as an immunogen, various tests may be performed in order to provide the necessary confirmation, cf. also the specifics set forth in the examples herein. In this context, reference is also made to the discussion of identification of useful IL5 analogues in WO 00/65058—this disclosure may be used for verifiction of the usefulness of an analogue (IL5 derived or not) subject to the present inventive technology.

Preferred multimers that may be subjected to the technology of the present invention are IL5 and TNFα.

IL5 Based Constructs

For hIL5 it has been found that constructs that mimic the natural hIL5 dimer structure and at the same time include foreign T_{H} elements provide superior results compared to constructs based on the monomeric structure, e.g. over the constructs disclosed in WO 00/65058, especially when it comes to expression levels and antibody reactivity of antisera raised against the constructs.

Preferred constructs based on IL5 are those wherein the analogue is selected from the group consisting of

two complete IL5 monomers joined by a peptide linker that includes at least one MHC Class II binding amino acid sequence, and

two complete IL5 monomers joined by an inert peptide linker of which at least one IL5 monmer includes a heterologous MHC Class II binding amino acid sequence.

Such an analogue may have the linear structure IL-Lₘ-IL or ILₘ-Lᵢ-ILₙ or IL-Lᵢ-ILₘ or IL-Lᵢ-ILₘ or ILₘ-Lₘ-ILₙ wherein “IL” is the complete amino acid sequence of monomeric mature IL5, “ILₘ” and “ILₙ”, which may be identical or non-identical, designate a substantially complete amino acid sequence of monomeric mature IL5 including a heterologous MHC Class II binding amino acid sequence, “Lₘ” is a peptide linker including or contributing to at least one MHC Class II binding amino acid sequence in the analogue, and “Lᵢ” is an inert peptide linker that does not include or contribute to any MHC Class II binding amino acid sequence in the analogue. It is especially preferred that Lₘ, ILₘ and ILₙ comprise the P2 and/or P30 epitopes of tetanus toxoid or comprises a PADRE, and Lᵢ is a di-glycine linker. However Lᵢ may be any non-immunogenic linker peptide that does not give rise to MHC Class II binding sequences.

Most preferred embodiments are hIL5 analogues having the mature amino acid sequence set forth in any one of SEQ ID NOs: 9, 11, 13 and 15.

TNFα Background

Tumour necrosis factor (TNF, TNFα, cachectin, TNFSF2) is a potent paracrine and endocrine mediator of inflammatory and immune functions. TNFα is cytotoxic for many cells especially in combination with gamma-interferon. TNFα was initially identified in 1975 and demonstrated to initiate tumor necrosis and regression. The anti-cancer effect has later been investigated in detail, but the treatment has not been a success as cancer therapy, although there are still cancer trials using TNFα running. TNFα was later discovered as the cause of cachexia and it was discovered that TNF exerts its function through a receptor-mediated process. Two different TNFα receptors (TNFR55 and TNFR75) have been identified that mediate cytotoxic and inflammatory effects of TNFα. TNFα induces and perpetuates inflammatory processes during chronic inflammatory diseases like rheumatoid arthritis (RA) and is suspected to have a critical role in allergies and psoriasis. Blocking of the TNFα0 signal by soluble receptors, receptor-specific inhibitors, down-regulation of TNFα production or monoclonal anti-TNFα antibodies are attractive therapy forms to adverse the biological effects of TNFα up-regulation and signaling.

It is evident from the results obtained from treatment with soluble TNFα receptors and monoclonal anti-TNFα antibodies that anti-TNFα therapy is a success in several diseases, like RA and Crohn's disease. The anti-TNFα treatment is both considered safe and effective.

To date, two TNFα antagonists, Remicade (Infliximab, Centocor/Johnson&Johnson) and Enbrel (Etanercept, Immunex) have been approved for clinical use.

Remicade is a chimeric mouse-human monoclonal IgG1 antibody directed against soluble and cell associated TNFα. Remicade blocks the binding of TNF with its endogenous cell surface TNFα receptor. The Food and Drug Administration (FDA) approved Remicade in October 1998 for use in moderate to severe or fistulizing Crohn's Disease refractory to conventional therapies. The indication was extended to include adjunctive use with methotrexate in rheumatoid arthritis refractory to methotrexate therapy alone and in July 2002 maintenance therapy in Crohn's disease.

Enbrel is a recombinant protein consisting of the extracellular portion of the human TNFα receptor fused to the Fc portion of human IgGi. Enbrel inhibits TNFα activity by serving as a decoy TNFα receptor. FDA approved Enbrel for use in rheumatoid arthritis in November 1998. More than 350.000 patients have been treated with these TNFα antagonists. Review of clinical efficacy and safety information of these agents are performed continuously and although infections and other immune-related adverse events remain a major concern for TNFα antagonists, recent safety evaluation of post-marketing experience performed by the FDA and the Committee for Proprietary Medicinal Products (CPMP) states that anti-TNFα therapies have a favorable risk-benefit balance although labelling changes, including changes on serious infections have been required.

Compared with the established anti-TNFα therapies, the presently suggested TNFα immunotherapy has the advantages of microgram amount vaccinations and less frequent injections to keep a high anti-TNFα in vivo titer compared with large infusions of monoclonal antibodies. The positive consequences are a lower risk for side effects and less expensive therapy. It is also believed that a natural polyclonal antibody response will act as a more efficient down-regulator of TNFα than other anti-TNFα therapies.

TNFα is translated as a 233 amino acid precursor protein and secreted as a trimeric type II transmembrane protein, which is cleaved by specific metalloproteases to a trimeric soluble protein where each identical monomeric subunit consists of 157 amino acids (the amino acid sequence of which is set forth in SEQ ID NO: 17). Human TNFα is non-glycosylated while murine TNFα has a single N-glycosylation site. The TNFα monomer has a molecular weight of 17 kDa while the trimer has a theoretical MW of 52 kDa, although a cross-linked trimer moves as 43 kDa in SDS-PAGE. TNFα contains two cysteines that stabilize the structure by forming an intramolecular disulphide bridge. Both the N and C-terminus of TNFα are important for the activity. Especially the C-terminus is sensitive as deletion of three, two and even one amino acid drastically decreases the solubility and bioactivity. The important amino acid is Leu157, which forms a stabilizing salt bridge between two monomers in the trimer. On the other hand deletion of the first eight amino acids increases the activity with a factor 1.5-5 while deletion of the first nine amino acids restores the full-length activity. TNFα is a well-studied protein and many of the intra- and inter-molecular interactions leading to trimer formation and receptor binding have been identified.

Hence, in nature, human TNFα (SEQ ID NO: 17) exists as both a dimer and a trimer, but the molecule is in both cases very suitable as a candidate target for the present invention.

TNFα Constructs

A preferred TNFα analogue is selected from the group consisting of 1) two or three complete TNF-α monomers joined end-to-end by a peptide linker, wherein at least one peptide linker includes at least one MHC Class II binding amino acid sequence, and 2) two or three complete TNF-α monomers joined end-to-end by an inert peptide linker, wherein at least one of the monomers include at least one foreign MHC Class II binding amino acid sequence or wherein at least one foreign MHC Class II binding amino acids sequence is fused to the N- or C-terminal monomer, optionally via an inert linker.

Particularly interesting are immunogenic TNFα molecules with high stability, since it has earlier been found by the inventors that monomeric TNFα constructs tend to be relatively unstable, cf., however, the discussion below.

Thus, this type of construct is very much in analogy to the above discussed IL5 constructs.

A gene encoding the 3 TNFα subunits linked together by epitopes and/or inert peptide linkers in a manner parallel to that discussed for IL5 has been produced. The goal has been to generate variant TNFα molecules with a conformation as close to the native TNFα trimer as possible. The variants are designed to efficiently elicit neutralizing antibodies against wtTNFα. The most suitable TNFα variants are soluble and stable proteins in the absence of detergents or other kinds of additives that could disrupt the protein conformation.

By expressing the three monomers linked together as one single polypeptide chain using linkers and T_{H} epitopes, it is intended to prepare TNFα variants that are more stable than previous variant TNFα immunogens. This will allow preservation of the TNFα structure, by introduction of the necessary T_{H} epitopes outside of stabilizing hydrogen bonds, salt bridges or disulfide bridges.

From the X-ray crystal structure of TNFα it is seen that the first 5 residues of the N terminal are too flexible to allow a structure determination. The C-terminus, however, is located close to the middle of the monomer interface and is less flexible. The distance between the C alpha atoms of Arg-6 and Leu-157 is 10 Å, which is the distance of 3-4 amino acid residues. Therefore it seems to be possible to link the monomeric subunits directly together, but since the C-terminals are located at a delicate site, it will probably be advantageous to use flexible linkers, e.g. glycine linkers, for this connection.

Five variants have until now been designed utilising the “monomerized trimer” approach. The control TNF_T0 (TNFα Trimer number 0, SEQ ID NO: 22) consists of the three monomers directly linked together by 2 separate glycine linkers (GlyGlyGly). TNF_{—}0 is designed so as to be as stable as the wild type trimeric protein. Of course, other inert flexible linkers known in the art of protein chemistry may be used instead of the above-mentioned glycine linkers, the important feature being that the flexible linker does not interfer adversely with the monomerized protein's capability of folding into a 3D structure that is similar to the 3D structure of physiologically active wtTNFα.

The TNF_{—}0 construct is expressed as a soluble protein in E. coli, and it has been used to prepare the exemplary construct TNF_T4 (SEQ ID NO: 57), which is a variant wherein the PADRE MHC Class II binding peptide (SEQ ID NO: 7) is introduced. In this construct, the ratio between monomeric units and foreign epitopes are thus 1 epitope per 3 monomers, instead of 1 epitope per monomer as is the case in prior art variants that relied on immunogenized monomeric proteins—this is also the case for SEQ ID NO: 55). This fact provides a potentially positive influence on the trimer stability. An offspring from this approach is the TNF_C2 variant (SEQ ID NO: 28, cf. below), which is a TNFα monomer with a PADRE epitope in the same position as in TNF_T4.

In parallel, the tetanus toxoid P2 and P30 epitopes (SEQ ID NOs: 3 and 5, respectively), have been used in the TNF_T1 and TNF_T2 variants (SEQ ID NOs: 49 and 51, respectively), containing one epitope in each linker region, and also in TNF_T3 (SEQ ID NO: 59) that contains one C-terminal epitope and one in the second linker region. Proteins are mostly folded from the N-terminal toward the C-terminal. The idea underlying TNF_T3 is that when the first two N-terminal domains fold up they will function as internal chaperones for the third domain (monomer), which is enclosed by epitopes.

It has been discovered that in addition to the technology described in detail above, where polymeric proteins are “monomerized”, TNFα (and possibly many other multimeric proteins) allows for the production of monomers that 1) include at least one stabilising mutation and/or 2) include at least one non-TNFα derived MHC Class II binding amino acid sequence, where these TNFα monomer variants are capable of folding correctly into a tertiary structure that subsequently allows for the formation of dimeric and trimeric TNFα proteins having a correct quarternary structure (as evidenced by these having receptor binding activity). Hence, in these constructs it has been possible to prepare variants of monomeric TNFα that does not necessarily need to be produced as monomerized trimers because the changes introduced in the monomer sequences introduce so limited disruption of the monomer's tertiary structure that a di- or trimer can be formed. In accordance with the ideas underlying the present invention, it has further been found that all such variant are expressible as soluble proteins from bacterial cells.

Hence, it is possible to prepare immunogenic TNFα variants according to the following strategies that can be combined and which may further be combined with the already discussed “monomerization approach” of the invention (since these particular modifications alle are non-destructive by nature):

The Flexible Loop Strategy

It has been discovered by the present inventors that insertion of the PADRE epitope (SEQ ID NO: 7) into loop 3 in position Gly108-Ala109 is a promising approach to prepare TNFα variants with a structure closely resembling the native TNFα molecule. It has been deduced from the TNFα crystal structure that a T_{H} epitope inserted directly into this position will not have any neighboring amino acid residues in close proximity to interact with. Studies with TNF34 (SEQ ID NO: 18), the first PADRE construct made according to this approach, has shown that approximately 5% of the expressed protein TNF34 was soluble in E. coli and 95% of the TNF34 was expressed as inclusion bodies when the bacterial host cells were grown at 37° C. but after an adaptation of the fermentation process where the fermentation temperature is 25° C., the yields of soluble protein from the fermentation is close to 100%. Hence, optimization of growth conditions increases the yield of soluble protein.

A number of other constructs have been prepared (TNF35-TNF39, SEQ ID NOs, 23, 24, 25, 26, and 27), where all of these solely rely on introduction of SEQ ID NO: 7 in the flexible loop 3.

Stability Enhancing Mutations

Introduction of T_{H} epitopes in the flexible loop 3 could potentially destabilize the structure of the TNFα variant. However, this potential destabilization can be counteracted by stabilization of the structure through introduction of cysteines that will form a disulfide bridge. A cystine pair in two different positions have until now been introduced in variants TNF34-A and TNF34-B (SEQ ID NOs: 29 and 30). Also, the flexible N-terminal (the first 8 amino acids) that is known to reduce the strength of the receptor interaction will be deleted in parallel, hence the variant TNF34-C (SEQ ID NO: 31). The disulfide bridge is introduced in the monomer for stabilization of the epitope insertion site together with the naturally occurring disulfide bridge (Cys-67 Cys-101). This strategy would also stabilise both a TNFα monomer as such and a monomerized di- or trimer.

Other Conststructs

Several different strategies have been employed in the design of variants that will be soluble expression products. TNFX1.1-2 (SEQ ID NOs: 32 and 33) are based on insertions of SEQ ID NO: 7 in the first loop of TNFα, where the insertion site is located at an intron position. In TNFX2.1 (SEQ ID NO: 34) an artificial “stalk” region is created containing an insertion of SEQ ID NO: 7.

Mutations of TNFα have revealed that large hydrophobic amino acid substitutions, pointing into the trimer interface, stabilize the trimer structure. TNFX3.1 and TNFX3.2 (SEQ ID NOs: 35 and 36) are proposals to stabilize the existing TNF34 variant. TNFX4.1 (SEQ ID NO: 37) uses di-glycine linkers to diminish structural constrains from the PADRE peptide on the overall TNF34 structure. TNFX5.1 (SEQ ID NO: 38) employs, as an insertion point, a loop structure found in the TNF family member BlyS. TNFX6.1-2, TNF7.1-2 and TNFX8.1 (SEQ ID NOs: 39, 40, 41, 42, and 43) are further variants. TNFX9.1 and TNFX9.2 (SEQ ID NOs: 44 and 45) are TNF34 variants that utilize identical overlapping TNFα sequences of 4-6 amino acids both pre and post the epitope. Finally, two variants (SEQ ID NOs: 46 and 47) are P2/P30 double variants in the same location as for the PADRE peptide in TNF34.

Further, from the crystal structure of TNFα it is observed that one stabilizing salt bridge is present within the TNFα monomer between the residues Lys-98 and Glu-116. The definition of a salt-bridge is an electrostatic interaction between side chain oxygens in Asp or Glu and positive charged atom side chain nitrogens in Arg, Lys or His with an interatomic distance less than 7.0 \dot{A}ngstrom. By site directed substitition mutations of Lys-98 with Arg or His at this position in combination with substitutions of Glu 116 with Asp, an improvement of the stability for this salt bridge and thereby the stability of the trimer molecule could be attained. It is also possible to exchange these salt bridges with disulphide bridges, in a manner described above.

It has been observed that murine TNFα is considerably more stable than the human TNFα regarding to solubility and proteolysis. Improvement of TNFα variants includes making site directed mutants so as to mimic murine TNFα crystal structure to obtain more proteolytically stable TNFα product.

From the x-ray structures of human and murine TNFα it is seen that the centre of the trimer (in the middle of the three TNFα monomers) is held together due to hydrophobic forces, whereas the top and the bottom of the trimer is connected due to natural occurring salt bridges. Therefore, by screening these salt bridges for stronger connections, the stability of the TNFα trimer would also be improved.

Finally, the preliminary results obtained with the TNFα variants of the present invention have surprisingly demonstrated that the variants are physiologically active, at least in the sense that they bind the TNF-receptors. However, since TNFα is a toxic protein, it is desired to prepare safe variants that will not cause severe side effects in subjects immunised with a vaccine according to the invention. Therefore, it is also an important embodiment of the invention to include detoxifying mutations in the constructs if these upon testing in relevant toxicity models are demonstrated to be of potential danger for vaccinated individuals.

A number of point mutations are known in the art to detoxify TNFα or at least reduce toxicity to a large extent. These point mutations will, if necessary, be introduced into the variants of the present invention. Expecially preferred mutations are substitutions corresponding to mature TNFα of Tyr-87 with a Ser, of Asp-143 with Asn, and of Ala-145 with Arg. Further, all effective mutations mentioned in Loetscher, H., Stueber, D., Banner, D., Mackay, F. and Lesslauer, W. 1993 JBC 268 (35) 26350-7, are also interesting embodiments in the detoxifying embodiments of the present invention.

In summary, the following specific TNFα variants have been prepared according to the present invention:

| | Last aa | First aa | Amino acids | | |
|---|---|---|---|---|---|
| TNF | before | after | deleted by | | Total |
| Constructs | epitope | epitope | insert | Mutations | length |
| TNF34 | 108 | 109 | — | | 170 |
| TNF35 | 106 | 107 | — | | 170 |
| TNF36 | 107 | 108 | — | | 170 |
| TNF37 | 108 | 110 | A | | 169 |
| TNF38 | 108 | 112 | AEA | | 167 |
| TNF39 | 106 | 112 | EGAEA | | 165 |
| TNFC2 | 170 | | — | GGG + PADRE added C- | 173 |
| | | | | terminally | |
| TNF34-A | 108 | 109 | — | Q67C, A111C | 170 |
| TNF34-B | 108 | 109 | — | A96C, I118C | 170 |
| TNF34-C | 108 | 109 | — | N-terminal VRSSSRTP | 162 |
| | | | | are deleted | |
| TNFX1.1 | 17 | 19 | A | | 169 |
| TNFX1.2 | 17 | 96 | ANPQA | | 165 |
| TNFX2.1 | 0 | 2 | V | PADRE added N-terminally | 170 |
| TNFX3.1 | 108 | 109 | — | L157F | 170 |
| TNFX3.2 | 108 | 109 | — | V49F | 170 |
| TNFX4.1 | 108 | 109 | — | Two glycines before | 174 |
| | | | | and after PADRE | |
| TNFX5.1 | 83 | 87 | AVS | | 167 |
| TNFX6.1 | 132 | 146 | SAEINRPDYLDFA | | 157 |
| TNFX6.2 | 135 | 146 | INRPDYLDFA | | 160 |
| TNFX7.1 | 63 | 77 | FKGQGCPSTHVLL | | 157 |
| TNFX7.2 | 71 | 85 | THVLLTHTISRIA | | 157 |
| TNFX8.1 | 126 | 140 | EKGDRLSAEINRP | | 157 |
| TNFX9.1 | 108 | 103 | — | The six amino acids | 176 |
| | | | | preceeding PADRE are | |
| | | | | duplicated after the | |
| | | | | epitope | |
| TNFX9.2 | 108 | 105 | — | The four amino acids | 174 |
| | | | | preceeding PADRE are | |
| | | | | duplicated after the | |
| | | | | epitope | |
| TNF34-P2-P30 | 108 | 109 | — | Both P2 and P30 | 194 |
| TNF34-P30-P2 | 108 | 109 | — | Both P30 and P2 | 194 |

The numbers used are from the N-terminal V in SEQ ID NO: 17 (that is, from amino acid no. 2 in SEQ ID NO: 17). Preceeding the N-terminal Valine is in some sequences a Methionine used for translation start.

The most preferred protein constructs of the invention are thus those represented by any one of SEQ ID NOs: 18, 23, 24, 25, 26, 27, 28, 29, 30, 31, 32, 33, 34, 35, 36, 37, 38, 39, 40, 41, 42, 43, 44, 45, 46, 47, 49, 51, 53, 55, 57, and 59, as well as any amino acid sequence derived therefrom that only include conservative amino acid changes or detoxifying amino acid changes thereof.

At any rate, it is an important embodiment that all of these TNFα variants discussed above are expressible as soluble proteins from bacterial cells such as E. coli.

The preferred vector is pET28b+ when the goal is expression from E.coli, p2Zop2F (SEQ ID NO: 60) is the vector used for insect cell expression, and pHP1 (or its commercially available “twin” pCI) is the vector used for expression in mammalian cells

General Therapies Provided by the Invention

The invention provides for methods whereby it becomes possible to down-regulate a particular polymeric protein in a very advantageous manner.

In general, there is provided a method for down-regulating a polymeric protein in an autologous host, the method comprising effecting presentation to the animal's immune system of an immunogenically effective amount of at least one immunogenic analogue of the invention. It is preferred that the autologous host is a mammal, most preferably a human being.

The method can be put into practice in a number of ways, of which administration of a protein vaccine is one choice, but also a nucleic acid vaccination strategy or a live vaccination strategy are of great interest.

Protein/Polypeptide Vaccination and Formulation

When effecting presentation of the analogues to an animal's immune system by means of administration thereof to the animal, the formulation of the polypeptide follows the principles generally acknowledged in the art.

Preparation of vaccines which contain peptide sequences as active ingredients is generally well understood in the art, as exemplified by U.S. Pat. No. 4,608,251; 4,601,903; 4,599,231; 4,599,230; 4,596,792; and 4,578,770, all incorporated herein by reference. Typically, such vaccines are prepared as injectables either as liquid solutions or suspensions; solid forms suitable for solution in, or suspension in, liquid prior to injection may also be prepared. The preparation may also be emulsified. The active immunogenic ingredient is often mixed with excipients which are pharmaceutically acceptable and compatible with the active ingredient. Suitable excipients are, for example, water, saline, dextrose, glycerol, ethanol, or the like, and combinations thereof. In addition, if desired, the vaccine may contain minor amounts of auxiliary substances such as wetting or emulsifying agents, pH buffering agents, or adjuvants which enhance the effectiveness of the vaccines; cf. the detailed discussion of adjuvants below.

The vaccines are conventionally administered parenterally, by injection, for example, either subcutaneously, intracutaneously, intradermally, subdermally or intramuscularly. Additional formulations which are suitable for other modes of administration include suppositories and, in some cases, oral, buccal, sublinqual, intraperitoneal, intravaginal, anal, epidural, spinal, and intracranial formulations. For suppositories, traditional binders and carriers may include, for example, polyalkalene glycols or triglycerides; such suppositories may be formed from mixtures containing the active ingredient in the range of 0.5% to 10%, preferably 1-2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10-95% of active ingredient, preferably 25-70%. For oral formulations, cholera toxin is an interesting formulation partner (and also a possible conjugation partner).

The polypeptides may be formulated into the vaccine as neutral or salt forms. Pharmaceutically acceptable salts include acid addition salts (formed with the free amino groups of the peptide) and which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The vaccines are administered in a manner compatible with the dosage formulation, and in such amount as will be therapeutically effective and immunogenic. The quantity to be administered depends on the subject to be treated, including, e.g., the capacity of the individual's immune system to mount an immune response, and the degree of protection desired. Suitable dosage ranges are of the order of several hundred micrograms active ingredient per vaccination with a preferred range from about 0.1 μg to 2,000 μg (even though higher amounts in the 1-10 mg range are contemplated), such as in the range from about 0.5 μg to 1,000 μg, preferably in the range from 1 μg to 500 μg and especially in the range from about 10 μg to 100 μg. Suitable regimens for initial administration and booster shots are also variable but are typified by an initial administration followed by subsequent inoculations or other administrations.

The manner of application may be varied widely. Any of the conventional methods for administration of a vaccine are applicable. These include oral application on a solid physiologically acceptable base or in a physiologically acceptable dispersion, parenterally, by injection or the like. The dosage of the vaccine will depend on the route of administration and will vary according to the age of the person to be vaccinated and the formulation of the antigen.

Some of the analogues of the vaccine are sufficiently immunogenic in a vaccine, but for some of the others the immune response will be enhanced if the vaccine further comprises an adjuvant substance.

Various methods of achieving adjuvant effect for the vaccine are known. General principles and methods are detailed in “The Theory and Practical Application of Adjuvants”, 1995, Duncan E. S. Stewart-Tull (ed.), John Wiley & Sons Ltd, ISBN 0-471-95170-6, and also in “Vaccines: New Generation Immunological Adjuvants”, 1995, Gregoriadis G et al. (eds.), Plenum Press, New York, ISBN 0-306-45283-9, both of which are hereby incorporated by reference herein.

It is especially preferred to use an adjuvant which can be demonstrated to facilitate breaking of the autotolerance to autoantigens; in fact, this is essential in cases where unmodified IL5 is used as the active ingredient in the autovaccine. Non-limiting examples of suitable adjuvants are selected from the group consisting of an immune targeting adjuvant; an immune modulating adjuvant such as a toxin, a cytokine, and a mycobacterial derivative; an oil formulation; a polymer; a micelle forming adjuvant; a saponin; an immunostimulating complex matrix (ISCOM matrix); a particle; DDA; aluminium adjuvants; DNA adjuvants; γ-inulin; and an encapsulating adjuvant. In general it should be noted that the disclosures above which relate to compounds and agents useful as first, second and third moieties in the analogues also refer mutatis mutandis to their use in the adjuvant of a vaccine of the invention.

The application of adjuvants include use of agents such as aluminium hydroxide or phosphate (alum), commonly used as 0.05 to 0.1 percent solution in buffered saline, admixture with synthetic polymers of sugars (e.g. Carbopol®) used as 0.25 percent solution, aggregation of the protein in the vaccine by heat treatment with temperatures ranging between 70° to 101° C. for 30 second to 2 minute periods respectively and also aggregation by means of cross-linking agents are possible. Aggregation by reactivation with pepsin treated antibodies (Fab fragments) to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be employed. Admixture with oils such as squalene and IFA is also preferred.

According to the invention DDA (dimethyldioctadecylammonium bromide) is an interesting candidate for an adjuvant as is DNA and γ-inulin, but also Freund's complete and incomplete adjuvants as well as quillaja saponins such as QuilA and QS21 are interesting as is RIBI. Further possibilities are monophosphoryl lipid A (MPL), the above mentioned C3 and C3d, and muramyl dipeptide (MDP).

Liposome formulations are also known to confer adjuvant effects, and therefore liposome adjuvants are preferred according to the invention.

Also immunostimulating complex matrix type (ISCOM® matrix) adjuvants are preferred choices according to the invention, especially since it has been shown that this type of adjuvants are capable of up-regulating MHC Class II expression by APCs. An ISCOM® matrix consists of (optionally fractionated) saponins (triterpenoids) from Quillaja saponaria, cholesterol, and phospholipid. When admixed with the immunogenic protein, the resulting particulate formulation is what is known as an ISCOM particle where the saponin constitutes 60-70% w/w, the cholesterol and phospholipid 10-15% w/w, and the protein 10-15% w/w. Details relating to composition and use of immunostimulating complexes can e.g. be found in the above-mentioned text-books dealing with adjuvants, but also Morein B et al., 1995, Clin. Immunother. 3: 461-475 as well as Barr I G and Mitchell G F, 1996, Immunol. and Cell Biol. 74: 8-25 (both incorporated by reference herein) provide useful instructions for the preparation of complete immunostimulating complexes.

Another highly interesting (and thus, preferred) possibility of achieving adjuvant effect is to employ the technique described in Gosselin et al., 1992 (which is hereby incorporated by reference herein). In brief, the presentation of a relevant antigen such as an antigen of the present invention can be enhanced by conjugating the antigen to antibodies (or antigen binding antibody fragments) against the Fcy receptors on monocytes/macrophages. Especially conjugates between antigen and anti-FcγRI have been demonstrated to enhance immunogenicity for the purposes of vaccination.

Other possibilities involve the use of the targeting and immune modulating substances (i.a. cytokines) mentioned in the claims as moieties for the protein constructs. In this connection, also synthetic inducers of cytokines like poly I:C are possibilities.

Suitable mycobacterial derivatives are selected from the group consisting of muramyl dipeptide, complete Freund's adjuvant, RIBI, and a diester of trehalose such as TDM and TDE.

Suitable immune targeting adjuvants are selected from the group consisting of CD40 ligand and CD40 antibodies or specifically binding fragments thereof (cf. the discussion above), mannose, a Fab fragment, and CTLA-4.

Suitable polymer adjuvants are selected from the group consisting of a carbohydrate such as dextran, PEG, starch, mannan, and mannose; a plastic polymer such as; and latex such as latex beads.

Yet another interesting way of modulating an immune response is to include the immunogen (optionally together with adjuvants and pharmaceutically acceptable carriers and vehicles) in a “virtual lymph node” (VLN) (a proprietary medical device developed by ImmunoTherapy, Inc., 360 Lexington Avenue, New York, N.Y. 10017-6501). The VLN (a thin tubular device) mimics the structure and function of a lymph node. Insertion of a VLN under the skin creates a site of sterile inflammation with an upsurge of cytokines and chemokines. T- and B-cells as well as APCs rapidly respond to the danger signals, home to the inflamed site and accumulate inside the porous matrix of the VLN. It has been shown that the necessary antigen dose required to mount an immune response to an antigen is reduced when using the VLN and that immune protection conferred by vaccination using a VLN surpassed conventional immunization using Ribi as an adjuvant. The technology is i.a. described briefly in Gelber C et al., 1998, “Elicitation of Robust Cellular and Humoral Immune Responses to Small Amounts of Immunogens Using a Novel Medical Device Designated the Virtual Lymph Node”, in: “From the Laboratory to the Clinic, Book of Abstracts, October 12^{th}-15^{th} 1998, Seascape Resort, Aptos, Calif.”.

It is expected that the vaccine should be administered at least once a year, such as at least 1, 2, 3, 4, 5, 6, and 12 times a year. More specifically, 1-12 times per year is expected, such as 1, 2, 3, 4, 5, 6, 7, 8, 9, 10, 11, or 12 times a year to an individual in need thereof. It has previously been shown that the memory immunity induced by the use of the preferred autovaccines according to the invention is not permanent, and therefor the immune system needs to be periodically challenged with the analogues.

Due to genetic variation, different individuals may react with immune responses of varying strength to the same polypeptide. Therefore, the vaccine according to the invention may comprise several different polypeptides in order to increase the immune response, cf. also the discussion above concerning the choice of foreign T-cell epitope introductions. The vaccine may comprise two or more polypeptides, where all of the polypeptides are as defined above.

The vaccine may consequently comprise 3-20 different analogues, such as 3-10 analogues. However, normally the number of analogues will be sought kept to a minimum such as 1 or 2 analogues.

Nucleic Acid Vaccination

As a very important alternative to classic administration of a peptide-based vaccine, the technology of nucleic acid vaccination (also known as “nucleic acid immunisation”, “genetic immunisation”, and “gene immunisation”) offers a number of attractive features.

First, in contrast to the traditional vaccine approach, nucleic acid vaccination does not require resource consuming large-scale production of the immunogenic agent (e.g. in the form of industrial scale fermentation of microorganisms producing proteins). Furthermore, there is no need to device purification and refolding schemes for the immunogen. And finally, since nucleic acid vaccination relies on the biochemical apparatus of the vaccinated individual in order to produce the expression product of the nucleic acid introduced, the optimum posttranslational processing of the expression product is expected to occur; this is especially important in the case of autovaccination, since, as mentioned above, a significant fraction of the original B-cell epitopes of the polymer should be preserved in the modified molecule, and since B-cell epitopes in principle can be constituted by parts of any (bio)molecule (e.g. carbohydrate, lipid, protein etc.). Therefore, native glycosylation and lipidation patterns of the immunogen may very well be of importance for the overall immunogenicity and this is expected to be ensured by having the host producing the immunogen.

It should be noted that the enhanced expression levels observed with the presently disclosed analogues is very important for efficacy of DNA vaccination, since the in vivo expression level is one of the determining factors in the immunogenic efficacy of a DNA vaccine

Hence, a preferred embodiment of the invention comprises effecting presentation of the analogue of the invention to the immune system by introducing nucleic acid(s) encoding the analogue into the animal's cells and thereby obtaining in vivo expression by the cells of the nucleic acid(s) introduced.

In this embodiment, the introduced nucleic acid is preferably DNA which can be in the form of naked DNA, DNA formulated with charged or uncharged lipids, DNA formulated in liposomes, DNA included in a viral vector, DNA formulated with a transfection-facilitating protein or polypeptide, DNA formulated with a targeting protein or polypeptide, DNA formulated with Calcium precipitating agents, DNA coupled to an inert carrier molecule, DNA encapsulated in a polymer, e.g. in PLGA (cf. the microencapsulation technology described in WO 98/31398) or in chitin or chitosan, and DNA formulated with an adjuvant. In this context it is noted that practically all considerations pertaining to the use of adjuvants in traditional vaccine formulation apply for the formulation of DNA vaccines. Hence, all disclosures herein which relate to use of adjuvants in the context of polypeptide based vaccines apply mutatis mutandis to their use in nucleic acid vaccination technology.

As for routes of administration and administration schemes of polypeptide based vaccines which have been detailed above, these are also applicable for the nucleic acid vaccines of the invention and all discussions above pertaining to routes of administration and administration schemes for polypeptides apply mutatis mutandis to nucleic acids. To this should be added that nucleic acid vaccines can suitably be administered intraveneously and intraarterially. Furthermore, it is well-known in the art that nucleic acid vaccines can be administered by use of a so-called gene gun, and hence also this and equivalent modes of administration are regarded as part of the present invention. Finally, also the use of a VLN in the administration of nucleic acids has been reported to yield good results, and therefore this particular mode of administration is particularly preferred.

Furthermore, the nucleic acid(s) used as an immunization agent can contain regions encoding the moieties specified in the claims, e.g. in the form of the immunomodulating substances described above such as the cytokines discussed as useful adjuvants. A preferred version of this embodiment encompasses having the coding region for the analogue and the coding region for the immunomodulator in different reading frames or at least under the control of different promoters. Thereby it is avoided that the analogue or epitope is produced as a fusion partner to the immunomodulator. Alternatively, two distinct nucleotide fragments can be used, but this is less preferred because of the advantage of ensured co-expression when having both coding regions included in the same molecule.

Accordingly, the invention also relates to a composition for inducing production of antibodies against IL5, the composition comprising

a nucleic acid fragment or a vector of the invention (cf. the discussion of nucleic acids and vectors below), and

a pharmaceutically and immunologically acceptable vehicle and/or carrier and/or adjuvant as discussed above.

Under normal circumstances, the nucleic acid is introduced in the form of a vector wherein expression is under control of a viral promoter. For more detailed discussions of vectors and DNA fragments according to the invention, cf. the discussion below. Also, detailed disclosures relating to the formulation and use of nucleic acid vaccines are available, cf. Donnelly J J et al, 1997, Annu. Rev. Immunol. 15: 617-648 and Donnelly J J et al., 1997, Life Sciences 60: 163-172. Both of these references are incorporated by reference herein.

Live Vaccines

A third alternative for effecting presentation of the analogues of the invention to the immune system is the use of live vaccine technology. In live vaccination, presentation to the immune system is effected by administering, to the animal, a non-pathogenic microorganism that has been transformed with a nucleic acid fragment encoding an analogue of the invention or with a vector incorporating such a nucleic acid fragment. The non-pathogenic microorganism can be any suitable attenuated bacterial strain (attenuated by means of passaging or by means of removal of pathogenic expression products by recombinant DNA technology), e.g. Mycobacterium bovis BCG., non-pathogenic Streptococcus spp., E. coli, Salmonella spp., Vibrio cholerae, Shigella, etc. Reviews dealing with preparation of state-of-the-art live vaccines can e.g. be found in Saliou P, 1995, Rev. Prat. 45: 1492-1496 and Walker PD, 1992, Vaccine 10: 977-990, both incorporated by reference herein. For details about the nucleic acid fragments and vectors used in such live vaccines, cf. the discussion below.

As an alternative to bacterial live vaccines, the nucleic acid fragment of the invention discussed below can be incorporated in a non-virulent viral vaccine vector such as a vaccinia strain or any other suitable pox virus.

Normally, the non-pathogenic microorganism or virus is administered only once to the animal, but in certain cases it may be necessary to administer the microorganism more than once in a lifetime in order to maintain protective immunity. It is even contemplated that immunization schemes as those detailed above for polypeptide vaccination will be useful when using live or virus vaccines.

Alternatively, live or virus vaccination is combined with previous or subsequent polypeptide and/or nucleic acid vaccination. For instance, it is possible to effect primary immunization with a live or virus vaccine followed by subsequent booster immunizations using the polypeptide or nucleic acid approach.

The microorganism or virus can be transformed with nucleic acid(s) containing regions encoding the moieties mentioned above, e.g. in the form of the immunomodulating substances described above such as the cytokines discussed as useful adjuvants. A preferred version of this embodiment encompasses having the coding region for the analogue and the coding region for the immunomodulator in different reading frames or at least under the control of different promoters. Thereby it is avoided that the analogue or epitopes are produced as fusion partners to the immunomodulator. Alternatively, two distinct nucleotide fragments can be used as transforming agents. Of course, having the adjuvating-moieties in the same reading frame can provide, as an expression product, an analogue of the invention, and such an embodiment is especially preferred according to the present invention.

Combination Treatment

One especially preferred mode of carrying out the invention involves the use of nucleic acid vaccination as the first (primary) immunization, followed by secondary (booster) immunizations with a polypeptide based vaccine or a live vaccines as described above.

Use of the Method of the Invention in Disease Treatment

The precise choice of treatment regimen depends on the choice of multimeric protein to target. For instance, when targeting IL5 all conditions discussed in WO 00/65058 are relevant, and when the target is TNFα the diseases/conditions that are relevant are rheumatoid arthritis, juvenile chronic arthritis, spondylarthropathies, polymyositis, dermatomyositis, vasculitis, psoriasis (plaque) and psoriatic arthritis, Mb. Crohn, chronic obstructive pulmonary disorder, myelodysplastic syndrome, uveitis in rheumatoid arthritis, acute pulmonary dysfunction, asthma, Wegener's granulomatosis, irritable bowel disease, temporomandibular disorder (painful jaw joint), stomatitisosteoporosis, and cancer cachexia as well as other inflammatory diseases and other conditions generally appreciated in the art to be linked to the adverse effects of TNFα. It is therefore possible to treat or ameliorate symptoms that are associated with any of these diseases by employing the method of the invention for down-regulating activity of a multimeric protein.

Compositions of the Invention

The invention also pertains to compositions useful in exercising the method of the invention. Hence, the invention also relates to an immunogenic composition comprising an immunogenically effective amount of an analogue defined above, said composition further comprising a pharmaceutically and immunologically acceptable diluent and/or vehicle and/or carrier and/or excipient and optionally an adjuvant. In other words, this part of the invention concerns formulations of analogues, essentially as described hereinabove. The choice of adjuvants, carriers, and vehicles is accordingly in line with what has been discussed above when referring to formulation of the analogues for peptide vaccination.

The analogues are prepared according to methods well-known in the art. Longer polypeptides are normally prepared by means of recombinant gene technology including introduction of a nucleic acid sequence encoding the analogue into a suitable vector, transformation of a suitable host cell with the vector, expression of the nucleic acid sequence (by culturing the host cell under appropriate conditions), recovery of the expression product from the host cells or their culture supernatant, and subsequent purification and optional further modification, e.g. refolding or derivatization. Details pertaining to the necessary tools are found below under the heading “Nucleic acid fragments and vectors of the invention” but also in the examples.

Shorter peptides are preferably prepared by means of the well-known techniques of solid- or liquid-phase peptide synthesis. However, recent advances in this technology has rendered possible the production of full-length polypeptides and proteins by these means, and therefore it is also within the scope of the present invention to prepare the long constructs by synthetic means.

Nucleic Acid Fragments and Vectors of the Invention

It will be appreciated from the above disclosure that modified polypeptides can be prepared by means of recombinant gene technology but also by means of chemical synthesis or semisynthesis; the latter two options are especially relevant when the modification consists of or comprises coupling to protein carriers (such as KLH, diphtheria toxoid, tetanus toxoid, and BSA) and non-proteinaceous molecules such as carbohydrate polymers and of course also when the modification comprises addition of side chains or side groups to an polymer-derived peptide chain. These embodiments, are, as will be understood from the above, not the preferred ones.

For the purpose of recombinant gene technology, and of course also for the purpose of nucleic acid immunization, nucleic acid fragments encoding the analogues are important chemical products (as are their complementary sequences). Hence, an important part of the invention pertains to a nucleic acid fragment which encodes an analogue as described herein, i.e. a polymer derived artificial polymer polypeptide as described in detail above. The nucleic acid fragments of the invention are either DNA or RNA fragments.

Most preferred DNA fragment of the invention comprises a nucleic acid sequence selected from the group consisting of SEQ ID NO: 8, 10, 12, 14, 17, 48, 50, 52, 54, 56, and 58 or a nucleic acid sequence complementary to any of these.

The nucleic acid fragments of the invention will normally be inserted in suitable vectors to form cloning or expression vectors carrying the nucleic acid fragments of the invention; such novel vectors are also part of the invention. Details concerning the construction of these vectors of the invention will be discussed in context of transformed cells and microorganisms below. The vectors can, depending on purpose and type of application, be in the form of plasmids, phages, cosmids, mini-chromosomes, or virus, but also naked DNA which is only expressed transiently in certain cells is an important vector (and may be useful in DNA vaccination). Preferred cloning and expression vectors of the invention are capable of autonomous replication, thereby enabling high copy-numbers for the purposes of high-level expression or high-level replication for subsequent cloning.

The general outline of a vector of the invention comprises the following features in the 5′→3′ direction and in operable linkage: a promoter for driving expression of the nucleic acid fragment of the invention, optionally a nucleic acid sequence encoding a leader peptide enabling secretion (to the extracellular phase or, where applicable, into the periplasma) of or integration into the membrane of the polypeptide fragment, th nucleic acid fragment of the invention, and optionally a nucleic acid sequence encoding a terminator. When operating with expression vectors in producer strains or cell-lines it is for the purposes of genetic stability of the transformed cell preferred that the vector when introduced into a host cell is integrated in the host cell genome. In contrast, when working with vectors to be used for effecting in vivo expression in an animal (i.e. when using the vector in DNA vaccination) it is for security reasons preferred that the vector is not incapable of being integrated in the host cell genome; typically, naked DNA or non-integrating viral vectors are used, the choices of which are well-known to the person skilled in the art.

The vectors of the invention are used to transform host cells to produce the modified IL5 polypeptide of the invention. Such transformed cells, which are also part of the invention, can be cultured cells or cell lines used for propagation of the nucleic acid fragments and vectors of the invention, or used for recombinant production of the modified IL5 polypeptides of the invention. Alternatively, the transformed cells can be suitable live vaccine strains wherein the nucleic acid fragment (one single or multiple copies) have been inserted so as to effect secretion or integration into the bacterial membrane or cell-wall of the modified IL5.

Preferred transformed cells of the invention are microorganisms such as bacteria (such as the species Escherichia [e.g. E. colil], Bacillus [e.g. Bacillus subtilis], Salmonella, or Mycobacterium [preferably non-pathogenic, e.g. M. bovis BCG]), yeasts (such as Saccharomyces cerevisiae), and protozoans. Alternatively, the transformed cells are derived from a multicellular organism such as a fungus, an insect cell, a plant cell, or a mammalian cell. Most preferred are cells derived from a human being, cf. the discussion of cell lines and vectors below. Recent results have shown great promise in the use of a commercially available Drosophila melanogaster cell line (the Schneider 2 (S₂) cell line and vector system available from Invitrogen) for the recombinant production of IL5 analogues of the invention, and therefore this expression system is particularly preferred, and therefore this type of system is also a preferred embodiment of the invention in general.

For the purposes of cloning and/or optimized expression it is preferred that the transformed cell is capable of replicating the nucleic acid fragment of the invention. Cells expressing the nucleic fragment are preferred useful embodiments of the invention; they can be used for small-scale or large-scale preparation of the analogue or, in the case of non-pathogenic bacteria, as vaccine constituents in a live vaccine.

When producing the analogues of the invention by means of transformed cells, it is convenient, although far from essential, that the expression product is either exported out into the culture medium or carried on the surface of the transformed cell, since both of these options facilitate subsequent purification of the expression product.

When an effective producer cell has been identified it is preferred, on the basis thereof, to establish a stable cell line which carries the vector of the invention and which expresses the nucleic acid fragment encoding the modified IL5. Preferably, this stable cell line secretes or carries the IL5 analogue of the invention, thereby facilitating purification thereof.

In general, plasmid vectors containing replicon and control sequences that are derived from species compatible with the host cell are used in connection with the hosts. The vector ordinarily carries a replication site, as well as marking sequences which are capable of providing phenotypic selection in transformed cells. For example, E. coli is typically transformed using pBR322, a plasmid derived from an E. coli species (see, e.g., Bolivar et al., 1977). The pBR322 plasmid contains genes for ampicillin and tetracycline resistance and thus provides easy means for identifying transformed cells. The pBR plasmid, or other microbial plasmid or phage must also contain, or be modified to contain, promoters that can be used by the prokaryotic microorganism for expression.

Those promoters most commonly used in prokaryotic recombinant DNA construction include the B-lactamase (penicillinase) and lactose promoter systems (Chang et al., 1978; Itakura et al., 1977; Goeddel et al., 1979) and a tryptophan (trp) promoter system (Goeddel et al., 1979; EP-A-0 036 776). While these are the most commonly used, other microbial promoters have been discovered and utilized, and details concerning their nucleotide sequences have been published, enabling a skilled worker to ligate them functionally with plasmid vectors (Siebwenlist et al., 1980). Certain genes from prokaryotes may be expressed efficiently in E. coli from their own promoter sequences, precluding the need for addition of another promoter by artificial means.

In addition to prokaryotes, eukaryotic microbes, such as yeast cultures may also be used, and here the promoter should be capable of driving expression. Saccharomyces cerevisiase, or common baker's yeast is the most commonly used among eukaryotic microorganisms, although a number of other strains are commonly available. For expression in Saccharomyces, the plasmid YRp7, for example, is commonly used (Stinchcomb et al., 1979; Kingsman et al., 1979; Tschemper et al., 1980). This plasmid already contains the trpl gene which provides a selection marker for a mutant strain of yeast lacking the ability to grow in tryptophan for example ATCC No. 44076 or PEP4-1 (Jones, 1977). The presence of the trpl lesion as a characteristic of the yeast host cell genome then provides an effective environment for detecting transformation by growth in the absence of tryptophan.

Suitable promoting sequences in yeast vectors include the promoters for 3-phosphoglycerate kinase (Hitzman et al., 1980) or other glycolytic enzymes (Hess et al., 1968; Holland et al., 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also ligated into the expression vector 3′ of the sequence desired to be expressed to provide polyadenylation of the mRNA and termination.

Other promoters, which have the additional advantage of transcription controlled by growth conditions are the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Any plasmid vector containing a yeast-compatible promoter, origin of replication and termination sequences is suitable.

In addition to microorganisms, cultures of cells derived from multicellular organisms may also be used as hosts. In principle, any such cell culture is workable, whether from vertebrate or invertebrate culture. However, interest has been greatest in vertebrate cells, and propagation of vertebrate in culture (tissue culture) has become a routine procedure in recent years (Tissue Culture, 1973). Examples of such useful host cell lines are VERO and HeLa cells, Chinese hamster ovary (CHO) cell lines, and W138, BHK, COS-7 293, Spodoptera frugiperda (SF) cells (commercially available as complete expression systems from i.a. Protein Sciences, 1000 Research Parkway, Meriden, Conn. 06450, U.S.A. and from Invitrogen), and MDCK cell lines. In the present invention, an especially preferred cell line the insect cell line S₂, available from Invitrogen, PO Box 2312, 9704 CH Groningen, The Netherlands.

Expression vectors for such cells ordinarily include (if necessary) an origin of replication, a promoter located in front of the gene to be expressed, along with any necessary ribosome binding sites, RNA splice sites, polyadenylation site, and transcriptional terminator sequences.

For use in mammalian cells, the control functions on the expression vectors are often provided by viral material. For example, commonly used promoters are derived from polyoma, Adenovirus 2, and most frequently Simian Virus 40 (SV40) or cytomegalovirus (CMV). The early and late promoters of SV40 virus are particularly useful because both are obtained easily from the virus as a fragment which also contains the SV40 viral origin of replication (Fiers et al., 1978). Smaller or larger SV40 fragments may also be used, provided there is included the approximately 250 bp sequence extending from the HindIII site toward the BglI site located in the viral origin of replication. Further, it is also possible, and often desirable, to utilize promoter or control sequences normally associated with the desired gene sequence, provided such control sequences are compatible with the host cell systems.

An origin of replication may be provided either by construction of the vector to include an exogenous origin, such as may be derived from SV40 or other viral (e.g., Polyoma, Adeno, VSV, BPV) or may be provided by the host cell chromosomal replication mechanism. If the vector is integrated into the host cell chromosome, the latter is often sufficient.

### EXAMPLE 1

### Design of 4 New Two-Epitope (P2+P30) Monomer IL5 Constructs

IL5 is an anti-parallel homo-dimmer, in which the C termini and N termini of the monomers are located closely in the molecule. This opens for the possibility of linking the two monomers into a single monomer, closely resembling the wild-type dimer quarternary structure.

We have approached this using either the p2/P30 epitopes as linker or by inserting a di-glycine linker as described previously in Li et al. 1997, PNAS USA 94(13): 6694-9.

The native hIL5 encoding DNA molecule used in all the research work was purchased from R&D systems (BBG16). This DNA sequence did not include the hIL5 leader sequence; hence was added a synthetic DNA sequence encoding the natural hIL5 leader peptide. The sequences encoding the P2 and P30 T cell epitopes are derived from tetanus toxoid. These sequences were inserted into the native sequence of the gene thus providing the immunogenic variants of IL5. The insertions are made preserving the reading frame in the IL5 gene.

The cloning strategy for making the variants is based on elongation of primers or DNA fragments with sequence overlap. First, two sets of primers with complementary 5′ ends making up the insertion are elongated in two separate PCR reactions using the wt IL5 DNA molecule as template and a flanking vector primer. Thereafter, these two double stranded fragments, which accordingly also have complementary 5′ ends, are annealed and elongated to include the complete insert in a second PCR. Finally, the fragment is amplified using the flanking primers. These inserts are then digested with the appropriate endonucleases, as is the vector and vector and inserts are ligated together. This procedure is a modification of the “splice by overlap extension” procedure described by Horton et al. 1989 and outlined in Current protocols in molecular biology (pp. 8.5.7-9) “Introduction of a point mutation by sequential PCR steps” by Ausabel et al.

Standard molecular biological techniques and DNA manipulations such as restriction enzyme digests, argarose gel electrophoresis, growth and storage of the E. coli cells were performed using standard molecular biological techniques described in the laboratory manual Sambrook, J., Fritsch, E. F. & Maniatis, T. 1989 and using the M&E standard protocols

### EXAMPLE 2

### hIL5.34 and hIL5.35

In order to have the T-cell epitopes internally in the molecule, P2 and P30 are inserted head to tail as a linker between the two IL5 monomers thereby giving rise to two constructs hIL5-P30-P2-hIL5 (hIL5.34, mature peptide in SEQ ID NOs: 5 and 6) and hIL5-P2-P30-hIL5 (hIL5.35, mature peptide in SEQ ID NOs: 7 and 8) —both DNA constructs encode the natural IL5 leader sequence, resulting in a mature expression product of 266 amino acids. The translation products resulting from these designs are intended to fold into a “monomeric IL5 dimer”, i.e. a monomeric molecule that has a tertiary structure that very much resembles the complete 3-dimensional structure of dimeric IL5.

### EXAMPLE 3

### hIL5.36 & hIL5.37

Based on the previous successful generation of a biologically active monomer “ILS dimer mimic” by insertion of a di-glycine-linker by J. Li et al., similar, but immunogenic, construct with the addition of T-cell epitopes were designed. The variant hIL5.36 thus has the structure of the mature peptide in SEQ ID NOs: 9 and 10 and variant hIL5.37 has the structure of the mature peptide in SEQ ID NOs: 11 and 12. Both these constructs encode a natural IL5 leader sequence followed by the first 4 amino acids in IL5 that in turn is followed by the first inserted epitope—the other epitope is positioned in the C-terminus.

There are 2 main reasons that the N-terminal epitope is not positioned N-terminally to the complete IL5 sequence in these two constructs instead of aiming at preserving the hIL5 sequence. By using the natural hIL5 leader peptide together with the N-terminus of hIL5 we ensure that the leader peptide is cleaved off correctly. And, since the N-terminus in IL5 constitutes a flexible region, it is not of significance for preservation of 3-dimensional structure of the resulting construct.

The translation products resulting from these designs are seem to fold into a “monomeric ILS dimer” as described in Example 2.

### EXAMPLE 4

### Expression Levels

The above described human IL5 analogues have been inserted into multiple vectors, used for construction, DNA vaccination and, recombinant expression in insect-, mammalian- or E. coli cells using standard methods in the art.

Especially, using standard expression systems and protocols in COS cells (transient expression) and in S₂ cells, it was found that the expression levels were even better than those obtained with constructs encoding IL5 wildtype protein and the expression levels also exceeded those obtained when expressing the hIL5 variants disclosed in WO 00/65058.

### EXAMPLE 5

### Induction of Anti-IL5 Cross-Reactive Antibodies

The presently disclosed analogues of human IL5 where used in standard immunization protocols of mice. In brief, mice were immunized with the above-described variants from examples 2 and 3. The murine anti hIL5 antibodies were isolated via immunoaffinity chromatography and their anti-hIL5 activity was compared to that of murine antibodies raised against wild-type hIL5. The results indicated that the antibodies were higher titered and also of higher affinity than antibodies against the analogues taugth in WO 00/65058.

Preliminary results also indicate that the multimer mimics according to the present invention have preserved at least some of the IL5 specific activity.

### EXAMPLE 6

### Preparation of TNFα Variants

A synthetic DNA sequence “SMTNFWT3” (SEQ ID NO: 16) encoding the wild type human TNFα monomer polypeptide (SEQ ID NO: 17) was delivered as a ligation product from Entelechon GmbH. The DNA sequence of the human hTNFα was optimised for expression in E. coli according to the Codon Usage Database by exclusion of all codons with a frequency in E. coli of less than 10%. Further, the sequence was designed to include a 5′ NcoI restriction site for subsequent cloning steps.

The SMTNFWT3 ligation product was introduced into the pCR 4 TOPO Blunt vector and E. coli DH10B cells were transformed. Plasmid DNA from 10 of the resulting SMTNFWT3TOPO clones was purified and five clones containing the expected fragment (when analysed by Restriction Enzyme (RE) digest) were selected.

The NcoI/EcoRI DNA fragments from the five potentially correct SMTNFWT3TOPO clones were isolated and transferred to the pET28b(+) vector and sequence determined. Insertions, deletions or substitutions were identified in four clones whereas one clone appeared to be correct. The correct construct—SMTNFWT3pET28 was subsequently used as template for the generation of all single TNFα variants.

### EXAMPLE 7

### TNF34 Construction

The PanDR epitope amino acid sequence (SEQ ID NOs: 7 and 20) was manually “reverse-translated” to a DNA sequence (SEQ ID NO: 19) optimised for expression in E. coli, see below, and inserted in loop 3 of TNFα by SOE PCR.

The resulting construct (a DNA sequence encoding SEQ ID NO: 18) was placed in the pET28b+ vector to generate TNF34-pET28b+.

### EXAMPLE 8

### Monomerized Trimer Construction

The monomerized trimer constructs are based on 3 TNFα encoding regions, separated by either a tri-glycine linker and/or an epitope encoding region.

The TNF-α gene was synthesized as three separate entities. The three fragments were assembled by SOE PCR, and the assembled gene (SEQ ID NO: 21) was cloned into pCR2.1-TOPO. After sequence verification, a correct clone was isolated. The hTNFT_{—}0 gene (SEQ ID NO: 21 encoding TNFα-GlyGlyGly-TNFα-GlyGlyGly-TNFα, SEQ ID NO: 22, i.e. 3 copies of SEQ ID NO: 17 separated by two tri-glycine linkers) was then transferred to pET28b+ to generate hTNFT_{—}0-pET28b+. A correct clone was isolated, sequence verified and transformed into E. coli lines BL21-STAR, BL21-GOLD and HMS174.

hTNFT_{—}0-pET28b+ was used as template to generate the following four monomerized trimer variants: hTNFT_{—}1, hTNFT_{—}2, hTNFT_{—}3 and hTNFT_{—}4 (SEQ ID NOs: 49, 51, 57, and 59) by SOE PCR. A further variant (SEQ ID NO: 53) can be made in a similar way.

hTNFT_{—}1, hTNFT_{—}2 and hTNFT_{—}3 are variants including tetanus toxoid epitopes P2 and P30 (SEQ ID NOs: 3 and 5, respectively) that need to be assembled by two rounds of SOE PCR. hTNFT_{—}4 is a variant with a PADRE (SEQ ID NO: 7) insert and can be assembled by a single round of SOE PCR. A further variant (SEQ ID NO: 55) can be made in a similar way.

hTNFT_{—}4 was constructed by the above mentioned methods, and a correct clone of hTNFT_{—}4-pET28b+ was found in TOP 10 cells and the construct was transferred to BL21-STAR and HMS174 cells.

To generate hTNFT_{—}1, hTNFT_{—}2 and hTNFT_{—}3 the epitopes were inserted by SOE PCR in very small fragments of the trimer, which were inserted into hTNFT_{—}0-pET28b+ by RE cutting and ligation.

### EXAMPLE 9

### Stabilising TNF34 Mutants

To further stabilise the TNF34-pET28b+ variant described above, variants containing the introduction of an extra disulfide bridge as well as a deletion mutant were constructed. 3 different variants were constructed: TNF34-A-pET28b+ contains the substitutions Q67C and A111C, TNF34-B-pET28b+ contains A96C and I118C, and TNF34-C-pET28b+ that contains a deletion of the 8 most N-terminal amino acids—the amiono acid sequences of the expression products are set forth in SEQ ID NOs: 20, 30, and 31.

All 3 constructs were made using SOE PCR, and were cloned in BL21-STAR, BL21-GOLD and HMS174, followed by sequence verification.

### EXAMPLE 10

### Flexible Loop Variants

In order to find a variant that might exhibit improved characteristics compared to the TNF34-pET28b+ variant, constructs were made where the PADRE insert (SEQ ID NO: 7) is moved around in flexible loop 3 of the TNF-α molecule.

All of these: TNF35-pET28b+, TNF36-pET28b+, TNF37-pET28b+, TNF38-pET28b+, TNF39-pET28b+, and a variant with PADRE placed in the C terminus of the molecule; TNFC2-pET28b+, were made with SOE PCR technique and were cloned in BL21-STAR, BL21-GOLD and HMS174, followed by sequence verification. The amino acid sequences of the expression products are set forth in SEQ ID NOs: 23, 24, 24, 26, 27 and 28.

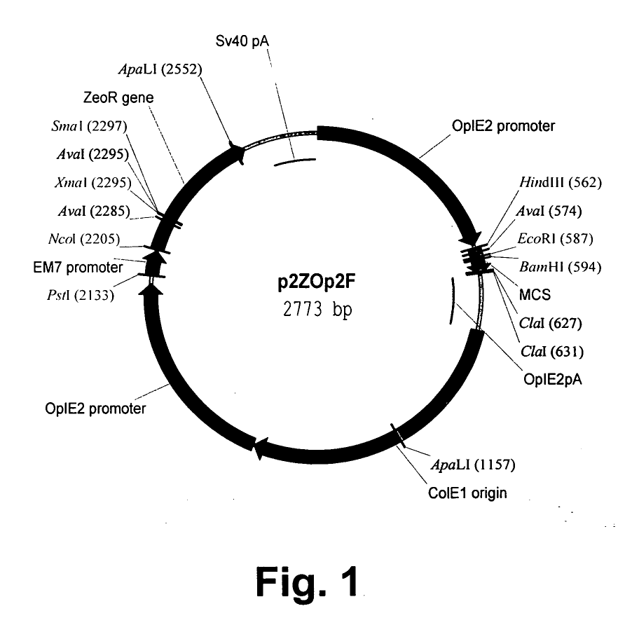

To also evaluate the possibility of using insect cells as expression system, TNFWT, TNF34, TNF35, TNF36, TNF37, TNF38, TNF39 and TNFC2 were transferred into the p2Zop2f vector (cf. FIG. 1), and expressed in S2 insect cells.

### EXAMPLE 11

### Other Constructs

A large number of further TNFα variants have been prepared, all termed TNFX, cf. above. The DNA encoding these variants has being made by SOE PCR, and cloned directly into pET28b+.

The correct TNFX clones have been transformed into BL21-STAR and HMS174, and subsequently sequence verified.

### EXAMPLE 12

### Periplasmic Expression

The LTB leader sequence has been added directly upstream of SEQ ID NO: 16 in TNF34-pET28b+, to target the expression to the periplasmic space.

### EXAMPLE 13

### Mammalian Expression

To test for expression in mammalian cells, SEQ ID NO: 16 and the DNA encoding TNF34 have been transferred to the pHP1 vector, which is a variant of the commercially available pCI vector (Promega Corporation). pHP1 includes a kanamycin resistance gene as marker instead of the AmpR gene of pCI.

### EXAMPLE 14

### Co-Expression of GroEL and GroE5

The expression of the E. coli chaperone complex, GroEL/ES, has previously been reported to increase the expression of soluble TNFα mutants (Jeong, W et al 1997, Biotechnology letters, vol 19, no 6 pp579-582). To test if the coexpression of GroEL/ES could improve the expression of the TNFα variants as herein described, a plasmid containing the GroEL/ES operon from E. coli under control of its natural promoter has been used. This plasmid has been co-transformed into HMS174 together with either DNA encoding wtTNFα, TNF34 or TNF37. Double transformants were selected by plating out on plates containing both Kanamycin and Carbecillin, which are the two relevant selection markers. Double transformants were then identified by RE analysis to test for the presence of both plasmids in the same clone.

In a pilot experiment, cells were grown at 37° C. to OD600=0.6-1 followed by a 30 min heatshock at 42° C. A control fraction of the cells were not heatshocked, and all cells were diluted 5 times into LB media containing 1 mM IPTG and grown ON at 25° C.

The cells were harvested and both supernatants and lysates were analysed for TNFα expression. Commassie staining was performed to evaluate the GroEL/ES expression.

In this experiment, no improvement by addition of chaperones was observed. This is mainly because we obtain almost 100% soluble material in this experiment, event in the absence of chaperones. We will however check the improvement on other variants of the invention if these appear to be less soluble variants.

### EXAMPLE 15

### E. coli Expression

Expression of soluble TNFα variants in three different E. coli strains has been tested in laboratory fermentors as well as in shake flasks. The fermentation equipment used was the Infors fermentor system with 1L working volume. The three E. coli strains tested were: HMS174, BL21 STAR and BL21 GOLD. The medium used for the fermentations was a defined minimal medium with glucose as the sole carbon source.

One of the primary objectives was to determine optimum fermentation process parameters (especially temperature and IPTG concentration) so as to optimise for expression of soluble TNFα variants.

Process Parameters:

| Parameter | Set point | Range | Action limit |
|---|---|---|---|
| pH | 7.0 | 6.5-7.5 | <6.4->7.6 |
| Temp. start | 37° C. | 36-38° C. | <36->38° C. |
| Temp. | 25° C. | 24-26° C. | <24->26° C. |
| induction | | | |
| DO₂ tension | 30% | 0-100% | >90% for more than 4 hours |
| Stirrer | 1000 RPM | 1000-1500 | — |

It has been found that one suitable scheme is the following: The IPTG concentration is 0,5 mM and the temperature at induction is lowered to 25° C. The total fermentation time is between 14 and 18 hours, including propagation, induction and protein production. The total fermentation time depends on the growth of the culture. OD600 start in the fermentor is typically between 0,1-0,3 (2-6 in the pre culture) as calculated from the OD in the inoculation culture. Induction of culture is performed at OD600=20±1-2 or nine to eleven hours after inoculation. Protein production then takes place for three to five hours.

Alternatively: Expression of TNF-α variant is accomplished by taking advantage of a low temperature culture to avoid intracellular precipitation of the variant protein to inclusion bodies. Growth of the culture to the wanted OD is done at the same temperature as the actual induction to avoid “shocks” to the cells by changing the temperature from the optimal growth temperature (37° C.) to the lower induction temperature (25° C.). By using this method it is believed that the only pressure imposed on the cells is the actual induction by IPTG—at any rate, this method has recently provided significantly improved yields of soluble expression product. By making a small over night culture and preparing the larger 1L LB medias a day in advance the generation of material in the large LB-cultures can be accomplished in approximately 9 hours while the actual induction period is done over night (in 16-20 hours). Hence, a preferred method can be described as follows: Expression of the TNFα variant is performed in 2×2 L baffled shake flasks containing 1 L LB media, each with the only modification to the above-mentioned method being that cells (BL21 STAR) are grown at 25° C. to an OD₄₃₆ of 0.7 after which the cells are induced with 1 mM IPTG and allowed to produce protein for 20 hours (still at 25° C.).

### EXAMPLE 16

### Selection Assays

A direct receptor ELISA together with a polyclonal ELISA and a cytotoxicity assay with KD-4 and Wehi cells are used as first line assays to screen and follow purification. Antibodies produced against TNFα variants are used to inhibit wtTNFα binding in both the receptor and the cytotoxic assay, to measure the antibody quality.

### EXAMPLE 17

### Purification Procedures

In this example, recombinant production and subsequent purification of one of the TNFα variants (TNF37) is described in detail. However, the purification procedure is the preferred one according to the present invention and will also be applicable (with small adjustments relevant for each variant) for other TNFα variants of the present invention.

An E. coli strain BL21 STAR/TNF37 colony from a LB-kanamycin plate (60 mg kanamycin/L LB media containing 1.5% Agar) is resuspended in 5 ml LB-media (60 mg kanamycin/L LB) and grown over night (16 hours) at 37° C. while shaking 220 RPM in a New Braunswick shaker.

2×2 ml of this culture is transferred to 2×1 L LB (60 mg kanamycin/L) in 2L baffled shake flasks and the cells are allowed to grow in a New Braunswick shaker at 220 RPM to OD₄₃₆=0.6-0.8. This step has been performed at the exemplary temperatures 37° C. and 25° C., but the temperature may be optimised for each culture.

1 ml 1 M IPTG is added to each flask and the cells are allowed to grow for 16-20 hours. Before induction, the temperature is adjusted to 25° C. if this is not already the fermentation temperature.

The cells are harvested in centrifuge tubes (500 ml) by centrifugation at 5000 RPM for 15 min using an SLA-3000 head in a Sorvall centrifuge.

The cells are transferred to one 500 ml pre-weight centrifuge tube using 0.9% NaCl and harvest cells by centrifugation as before.

The supernatant is discharged and the tube is weighed to determine the cell weight (should be 7-11 grams).

200 ml 50 mM Na₂HPO₄, pH=7.0 is added (if cells are re-suspended they should be used directly, otherwise it is possible to freeze).

Cell Disruption, Centrifugation, and Filtration

A mechanical disruption of the cells offer several advantages over enzymatic disruption in terms of efficiency, reliability and the ability to choose any buffer necessary in the following steps of the purification. The APV-1000 is kept cool during the operation by adding ice water to the sample-chamber before use and pas ice water through the machine between the two passages of sample. Centrifugation and filtration serves to remove any particles or aggregates from solution prior to chromatographic separation of the proteins. The cell disruption and HA-chromatography should be done the same day as this might minimize the apparent protease activity as a consequence of the separation from these in the chromatographic step. The procedure for disruption, centrifugation and filtration is as follows:

The carefully re-suspended cell material is transferred from to the cell-disrupter (APV-1000). The cell-suspension is carefull passed 2× through the disrupter (cooling on ice after each passage and passing ice water through the APV-1000 in between the passages) using 700 bars of backpressure (the solution ought to be clear at this point).

The disrupted cells are transferred to a 500 ml centrifuge tube and the cells are spun for 45 min at 10000 RPM in a Sorvall centrifuge using the SLA-3000 head.

The extract (approx 225 ml) is passed through a 0.22 μm filter.

Hydroxyapatite(HA) Chromatography

Hydroxyapatite Bio-Gel HTP Gel (BIO-RAD; catalog # 130-0420) is a crystalline form of calcium phosphate having proven itself as a unique tool in the separation of proteins such as monoclonal antibodies and other proteins otherwise not separable by other methods. However, in our experience the flow properties of the material are somewhat critical in that sense that a flow higher than 2 ml/min raises the pressure to an unacceptable high level. Also the material has collapsed several times when attempt has been made to regenerate with sodium hydroxide as recommended by the manufacturer.

Buffers and Column

Stock for buffer A+B: 1 M Na₂HPO₄×2H₂O, pH=7.0 (pH adjusted to 7 with HCl). Buffers A+B made from dilutions of stock.

Buffer A: 50 mM Na₂HPO₄×2H₂O, pH=7.0

Buffer B: 0.3 M Na₂HPO₄×2H₂O, pH=7.0

Column packed to approximately 50-60 ml with hydroxyapatite Bio-Gel HTP Gel (BIO-RAD; catalog # 130-0420) using a suspension in Buffer A and a XK 26/40 (Amersham Biosciences) column.

Chromatography Program

Purge system 20 ml at a flow of 30 ml/min.

Equilibration: 4 CV of Buffer A at a flow of 2 ml/min

Load sample through pump (inlet F on the BioCad) (approx 225+5-10 ml if the sample in the tubing is needed) at a flow of 2 ml/min.

Wash column with 1.5 CV Buffer A at a flow of 2 ml/min.

Elution: Elute protein with a gradient of 4 CV from 0% to 100% Buffer B at a flow of 2 ml/min.

Clean column with 2 CV Buffer B at a flow of 2 ml/min.

Re-equilibration with 4 CV Buffer A at a flow of 2 ml/min.

Select fractions, pool, and dialyse ON at 4° C. against 15× volume 20 mM Tris-HCl, 0.075 M NaCl, pH 8.0.

Selecting TNF37-Containing Fractions After HA Chromatography

The HA chromatography elution fraction profile basically consist of a “run through” fraction and one eluted peak that can be separated into several peaks. The TNF37-containing fractions has to be selected on the basis of a coomassie stained gel of the entire peak since a peak containing TNF37 is not directly identifiable. However, as a consequence of subsequent purification steps the selection of fractions at this point is less critical and it is possible to remove contaminants later in the procedure. Thus, a less conservative selection of fractions ensures maximum yield of variant.

Initially the “run through” was checked with “dot blots” for any TNF37. This gave a positive result that in theory should indicate that a significant part of the variant did not bind to the column. However, when the “run through” is subjected to the very efficient SP-sepharose Cation Exchange Chromatography (cf. next step) and the fractions are analysed with coomassie stained gels they do not contain any detectable TNF37-variant indicating some false positive reaction in the “dot blot” or a fraction of the variant that binds completely different to the SP-sepharose.

SP-Sepharose Cation Exchange Chromatography

SP-sepharose is a basic cation exchange step selected as consequence of the rather high calculated pI of 9.4 of the variant compared to the wtTNFα pI of 7.8. This increase in pI is a consequence of the 2 lysines introduced via the PADRE epitope. This chromatography is very efficient and fast for the TNF37 variant and is expected to be useful for a large number of other loop variants of TNFα.

The sample applied should have a lower conductivity than 8 mS/cm and pH should be at least 7.7 before continuing with SP-sepharose chromatography since variations from this in our experience has made the binding properties of the protein different from time to time.

Buffers and Column

Stocks to buffers A+B: 1 M Tris-HCl. pH =8.0.

Buffer A: 20 mM Tris-HCl, 0.075 M NaCl, pH=8.0.

Buffer B: 20 mM Tris-HCl, 1 M NaCl, pH=8.0.

Column packed to approximately 60 ml with SP-sepharose FF (Amersham Biosciences; catalogue # 17-0729-01) using a suspension in Buffer A and a XK 26/40 (Amersham Biosciences) column.

Chromatography Program

Purge system 20 ml at a flow of 30 ml/min

Equilibration: 4 CV of Buffer A at a flow of 4 ml/min.

Load sample through pump (inlet F on the BioCad) (Sample+10 ml if the sample in the tubing is needed) at a flow of 4 ml/min.

Wash column with 1.5 CV Buffer A at a flow of 4 ml/min.

Elution: Elute protein with a gradient of 4 CV from 0% to 100% Buffer B at a flow of 4 ml/min.

Clean column with 2 CV Buffer B at a flow of 4 ml/min.

Re-equilibration with 4 CV Buffer A at a flow of 4 ml/min.

Select fractions, pool, and dialyse ON at 4° C. against 15× volume 20 mM Tris-HCl, 0.075 M NaCl, pH 8.0.

Selecting TNF37 Containing Fractions After SP Sepharose Chromatography

The profile basically consists of a “run through” fraction and several protein containing peaks. However two peaks contains the variant with some contaminants. It is at this point important not to include to many fractions on the right side of peak two since this in our experience includes to many contaminants that are not easily removed in subsequent chromatographic steps.

Q-Sepharose Anion Exchange Chromatography

Q-sepharose is a basic anion exchange step selected for removing a major contaminant protein that with high reproducibility follows the purification of TNF37 including the HA-chromatography and SP-sepharose. The TNF37 variant itself does not bind to the column but the major unknown contaminant does. It is, however, possible to select fractions in a conservative fashion already in the SP-sepharose step in that way avoiding the contaminant. However, this compromises the yield of TNF37 variant compared to when the Q-sepharose is used in the procedure and since also other minor contaminants are removed in this step, it is preferred to include it in the total procedure. In conclusion the Q-sepharose step is important in the purification of variant 37 and offers an even better end product with a high yield.

Buffers and Column

Stocks to buffers A+B: 1 M Tris-HCl. pH=8.0.

Buffer A: 20 mM Tris-HCl, 0.075 M NaCl, pH=8.0.

Buffer B: 20 mM Tris-HCl, 1 M NaCl, pH=8.0.

Column packed to approximately 50-60 ml with Q-sepharose FF (Amersham Biosciences; catalogue # 17-0510-01) using a suspension in Buffer A and a XK 26/40 (Amersham Biosciences) column.

Chromatography Program

Purge system 20 ml at a flow of 30 ml/min.

Equilibration: 4 CV of Buffer A at a flow of 4 ml/min

Load sample through pump (inlet F on the BioCad) (Sample+10 ml if the sample in the tubing is needed) at a flow of 2 ml/min.

Wash column with 3 CV Buffer A at a flow of 4 ml/min.

Elution: Elute remaining protein with 2 CV 100% Buffer B at a flow of 4 ml/min.

Re-equilibration with 4 CV Buffer A at a flow of 4 ml/min.

Select fractions, pool and apply directly on SP-sepharose column.

The elution profile basically consists of a “Run through” fraction and several protein containing peaks. The “Run through” fraction can sometimes be divided into several purely resolved peaks which all contains the TNF37 variant and therefore all are pooled. This heterogeneity of the TNF37 is probably solved when the problem with the apparent proteolytic degradation is solved.

### EXAMPLE 18

### Immunisation Studies

Materials:

Saline (0,9% NaCl in sterile water, Fresenius Kabi Norge AS, Norway)

Complete Freund's Adjuvant (Sigma, F-5881, 39H8926)

Incomplete Freund's Adjuvant (Sigma, F-5506, 60K8937)

Alhydrogel 2% [10 mg Al/ml](Brenntag Biosector, Batch 96 (3176))

Adjuphos[5 mg Al/ml] (Brenntag Biosector, Batch 2 (8937))

Wild type human TNF (Invitrogen cat.no:10062-024).

KYM-1D4: Provided by A. Meager (A. Meager, J. Immunol. Methods 1991, 144:141-143)

WEHI 164 clone 13: Provided by T. Espevik (T. Espevik and J. Nissen-Myer, J. Immunol. Methods 1986, 95:99-105)

Tetrazolium salt (MTS, CellTiter 96 Aqueous one solution; Promega, G3581)

Rotating bar (Rotamix, Heto, Denmark)

Vortex (OLE DICH instrumentmakers ApS, Denmark)

Choice of Formulation/Adjuvant

The purified TNFα variant proteins (in 20 mM Tris-HCl, 0.075 M NaCl, pH 8.0) are diluted to 0,5 mg/ml with saline (0,9% NaCl), batched (375 μg/vial) and stored at −20° C. until used for immunizations.

For each TNF variant, immunizations are made with two adjuvants: 1) Complete Freund's Adjuvant (CFA, for the primary immunization) and Incomplete Freund's Adjuvant (IFA, for boost immunizations) and 2) Alhydrogel or Adjuphos (state-of-the-art Aluminium hydroxide and aluminium phosphate adjuvants, respectively)—these are used for both prime and boost injections.

Before primary immunization, a decision on the choice of either Alhydrogel or Adjuphos as adjuvant for the TNF variant is made. The adjuvant with the best ability to adsorb the TNF variant is chosen for further use in the immunization experiment. Two aliquots of the TNFα variant are mixed with an equal volume of Alhydrogel and Adjuphos in two vials. The vials are gently mixed at room temperature for 30 minutes on a rotating bar. Vials are then centrifuged at 13000 g for 15 minutes and supernatant is tested for the soluble TNF variant content on a gradient (4-12%) SDS gel. The adjuvant/variant aliquot containing the least free variant (i.e. where more variant has bound to aluminium-particles) is then selected as the best adjuvant.

Preparation of Antigen/Adjuvant Emulgate:

CFA/IFA emulgates are prepared through the following procedure:

Vials with TNFα variant [0,5 mg/ml] is thawed, transferred to a 10 ml sterile vial and mixed with an equal volume of CFA or IFA. The vial is then mixed further on a vortex at 3300 rpm for 30 minutes at 20° C.

Alhydrogel/Adjuphos emulgates are prepared through the following procedure:

Alhydrogel/Adjuphos are diluted to 1,4 mg Al/ml with saline. Vials with TNFα variant [0,5 mg/ml] is thawed, transferred to a 10 ml sterile vial and mixed with an equal volume of Alhydrogel [1,4 mg Al/ml] or Adjuphos [1,4 mg Al/ml]. The vial is then mixed further on a rotating bar for 30 minutes at 20° C.

Choice of Animal Model

Six—eight weeks old Balb/Ca female mice are repetitively immunized with TNFα variants. Blood samples are collected at different intervals and isolated sera are investigated for anti-wtTNFα antibody titers. Mice are ordered from Taconic Farms, Inc. Acquires M&B A/S, Denmark. Mice are housed at the animal facility of Pharmexa for one week before initiation of experiment.

Immunization Scheme and Dosage

Groups of 10+10 mice are immunized with each TNFα variant in CFA/IFA and Alhydrogel/Adjuphos respectively. 20+20 mice are used for immunization with wild type TNFα.

At the first immunization, 50 μg of protein in adjuvant will be injected subcutanously. All mice will receive additional booster immunizations subcutanously with 25 μg of protein in adjuvant 2, 6 and 10 weeks after the first immunization.

Blood samples will be collected immediately before the first immunization and 1 week after each boost immunization.

Assays Employed

Cytotoxicity bioassay using WEHI 164 clone 13- or KYM-1D4-cells: This assay is used to determine the toxicity of TNFα variants of the invention. Cells are cultured for 48 hours in the presence of titrated amounts of TNFα variants and cell death is determined by addition of Tetrazolium salt (MTS), which is bioreduced into a coloured formazan product by living cells. Cytotoxicity of TNFα variants are compared to that of human wild type TNFα.

Cytotoxicity-inhibition bioassay using WEHI 164 clone 13- or KYM-1D4-cells: This assay is used to investigate the ability of anti-sera raised in TNFα immunized mice to neutralize the cytotoxic effect of wild type TNFα. Cells are cultured for 48 hours with titrated amounts of anti-sera and a constant concentration of wild type human TNFα, which is sufficient to induce cell death in 50% of cells in the absence of anti-sera. Cell death is determined by MTS, as described above. Neutralization-ability of sera from TNFα variant-immunized mice are compared to sera obtained from mice immunized with human wild type TNFα.

In Vitro Studies

Cytotoxicity bioassay using WEHI 164 clone 13- or KYM-1D4-cells: Cytotoxicity-inhibition bioassay using WEHI 164 clone 13- or KYM-1D4-cells.

Criteria for Choice of Best Immunogenic Constructs

TNFα variants should display minimal cytotoxicity. Immunization of mice with TNFα variants should generate anti-sera with better or equal ability to neutralize human wild type TNFα-mediated cytotoxicity in WEHI- or KYM-1D4 cells as sera obtained from human wild type TNFα-immunized mice.

The present invention will now be further described by way of the following numbered paragraphs:

1. An immunogenic analogue of a human TNFα, wherein said analogue

a) includes substantial fragments of at least 2 monomeric units of human TNFα, wherein said substantial fragments are joined via peptide bonds through a peptide linker,

b) includes at least one MHC Class II binding amino acid sequence that is heterologous to human TNFα, and

c) can be produced as one single expression product from a cell harbouring an expression vector encoding the analogue.

2. The immunogenic analogue according to paragraph 1, wherein each of the substantial fragments displays a substantial fraction of B-cell epitopes found in the corresponding monomers when being part of the human TNFα protein.

3. The immunogenic analogue according to paragraph 2, wherein each of the substantial fragments displays essentially all B-cell epitopes found in the corresponding monomers when being part of human TNFα.

4. The immunogenic analogue according to paragraph 2 or 3, wherein an amino acid sequence derived from a monomeric unit is modified by means of amino acid insertion, substitution, deletion or addition so as to reduce toxicity of the analogue as compared to human TNFα and/or so as to introduce the MHC Class II binding amino acid sequence.

5. The immunogenic analogue according to any one of paragraphs 1-4, wherein each of the substantial fractions comprises essentially the complete amino acid sequence of each monomeric unit, either as a continuous sequence or as a sequence including inserts.

6. The immunogenic analogue according to any of the preceding paragraphs, wherein amino acid sequences of all monomeric units of human TNFα are represented in the analogue.

7. The immunogenic analogue according to paragraph 6 that includes the complete amino acid sequences of the monomers constituting human TNFα, either as unbroken sequences or as sequences including inserts.

8. The immunogenic analogue according to any one of the preceding paragraphs, wherein the peptide linker includes or contributes to the presence in the analogue of at least one MHC Class II binding amino acid sequence that is heterologous to human TNFα.

9. The immunogenic analogue according to any one of paragraphs 1-7, wherein the peptide linker is free of and does not contribute to the presence of an MHC Class II binding amino acid sequence in humans.

10. The immunogenic analogue according to any one of the preceding paragraphs wherein the MHC Class II binding amino acid sequence binds a majority of MHC Class II molecules in humans.

11. The immunogenic analogue according to any one of the preceding paragraphs, wherein the at least one MHC Class II binding amino acid sequence is selected from a natural T-cell-epitope and an artificial MHC-II binding peptide sequence.

12. The immunogenic analogue according to paragraph 10, wherein the natural T-cell epitope is selected from a Tetanus toxoid epitope such as P2 or P30, a diphtheria toxoid epitope, an influenza virus hemagluttinin epitope, and a P. falciparum CS epitope.

13. The immunogenic analogue according to any one of the preceding paragraphs, wherein the 3-dimensional structure of the complete human TNFα is essentially preserved.

14. The immunogenic analogue according to any one of the preceding paragraphs, wherein the analogue is selected from the group consisting of

two or three complete TNFα monomers joined end-to-end by a peptide linker, wherein at least one peptide linker includes at least one MHC Class II binding amino acid sequence,

two or three complete TNFα monomers joined end-to-end by an inert peptide linker, wherein at least one of the monomers include at least one foreign MHC Class II binding amino acid sequence or wherein at least one foreign MHC Class II binding amino acids sequence is fused to the N- or C-terminal monomer, optionally via an inert linker.

15. An immunogenic analogue of human TNFα, wherein the analogue includes at least one foreign MHC Class II binding amino acid sequence and further has the characteristic of being

a human TNFα monomer or an analogue according to any one of the preceding paragraphs, wherein has been inserted or in-substituted at least one foreign MHC Class II binding amino acid sequence into flexible loop 3, and/or

a human TNFα monomer or an analogue according to any one of the preceding paragraphs, wherein has been introduced at least one disulfide bridge that stabilises the TNFα monomer 3D structure, and/or

a human TNFα monomer or an analogue according to any one of the preceding paragraphs, wherein any one of amino acids 1, 2, 3, 4, 5, 6, 7, 8, and 9 in the amino terminus have been deleted, and/or

a human TNFα monomer or an analogue according to any one of the preceding paragraphs, wherein an inserted or in-substituted at least one foreign MHC Class II binding amino acid sequence into loop 1 in an intron position, and/or

a human TNFα monomer or an analogue according to any one of the preceding paragraphs, wherein at least one foreign MHC Class II binding amino acid sequence is introduced as part of an artificial stalk region in the N-terminus of human TNFα, and/or

a human TNFα monomer or an analogue according to any one of the preceding paragraphs, wherein at least one foreign MHC Class II binding amino acid sequence is introduced so as to stabilize the monomer structure by increasing the hydrophobicity of the trimeric interaction interface, and/or

a human TNFα monomer or an analogue according to any one of the preceding paragraphs, wherein at least one foreign MHC Class II binding amino acid sequence flanked by glycine residues is inserted or in-substituted in the TNFα amino acid sequence, and/or

a human TNFα monomer or an analogue according to any one of the preceding paragraphs, wherein at least one foreign MHC Class II binding amino acid sequence is inserted or in-substituted in the D-E loop, and/or

a human TNFα monomer or an analogue according to any one of the preceding paragraphs, wherein at least one foreign MHC Class II binding amino acid sequence is inserted or in-substituted between two identical subsequences of human TNFα, and/or

a human TNFα monomer or an analogue according to any one of the preceding paragraphs, wherein at least one salt bridge in human TNFα has been strengthened or substituted with a disulphide bridge, and/or

a human TNFα monomer or an analogue according to any one of the preceding paragraphs, wherein solubility and/or stability towards proteolysis is enhanced by introducing mutations that mimic murine TNFα crystalline structure, and/or

a human TNFα monomer or an analogue according to any one of the preceding paragraphs, wherein potential toxicity is reduced or abolished by introduction of at least one point mutation.

16. An immunogenic analogue according to paragraph 14 or 15, wherein the amino acid sequence of the analogue is selected from the group consisting of SEQ ID NO: 18, 23, 24, 25, 26, 27, 28, 29, 30, 31, 32, 33, 34, 35, 36, 37, 38, 39, 40, 41, 42, 43, 44, 45, 46, 47, 49, 51, 53, 55, 57, and 59, and any amino acid sequence that only include conservative amino acid changes thereof.

17. An immunogenic analogue according to any one of the preceding paragraphs which can be expressed as a soluble protein from bacterial cells.

18. A nucleic acid fragment that encodes an immunogenic analogue according to any one of the preceding paragraphs, or a nucleic acid fragment complementary thereto.

19. The nucleic acid fragment according to paragraph 18 that is a DNA fragment.

20. The nucleic acid fragment according to paragraph 18 which comprises a nucleic acid sequence selected from the group consisting of SEQ ID NO: 17, 48, 50, 52, 54, 56, and 58 or a nucleic acid sequence complementary thereto.

21. A method for down-regulating a human TNFα in a human, the method comprising effecting presentation to the immune system of an immunogenically effective amount of at least one immunogenic analogue according to any one of paragraphs 1-17.

22. The method according to paragraph 21, wherein presentation is effected by administering the immunogenic analogue according to any one of paragraphs 1-17 to the autologous host, optionally in admixture with an adjuvant.

23. The method according to paragraph 22, wherein the adjuvant is selected from the group consisting of an immune targeting adjuvant; an immune modulating adjuvant such as a toxin, a cytokine and a mycobacterial derivative; an oil formulation; a polymer; a micelle forming adjuvant; a saponin; an immunostimulating complex matrix (an ISCOM matrix); a particle; DDA; aluminium adjuvants; DNA adjuvants; γ-inulin; and an encapsulating adjuvant.

24. The method according to any one of paragraphs 21-23, wherein an immunogenically effective amount of analogue is administered to the animal via a route selected from the parenteral route such as the intradermal, the subdermal, and the intramuscular routes; the peritoneal route; the oral route; the buccal route; the sublinqual route; the epidural route; the spinal route; the anal route; and the intracranial route.

25. The method according to paragraph 24, wherein the effective amount is between 0.5 μg and 2,000 μg.

26. The method according to paragraph 24 or 25, which includes at least one administration per year, such as at least 2, at least 3, at least 4, at least 6, and at least 12 administrations per year.

27. The method according to paragraph 21, wherein presentation of the analogue to the immune system is effected by introducing nucleic acid(s) encoding the analogue into the animal's cells and thereby obtaining in vivo expression by the cells of the nucleic acid(s) introduced.

28. The method according to paragraph 27, wherein the nucleic acid(s) introduced is/are selected from naked DNA, DNA formulated with charged or uncharged lipids, DNA formulated in liposomes, DNA included in a viral vector, DNA formulated with a transfection-facilitating protein or polypeptide, DNA formulated with a targeting protein or polypeptide, DNA formulated with Calcium precipitating agents, DNA coupled to an inert carrier molecule, DNA encapsulated in chitin or chitosan, and DNA formulated with an adjuvant such as the adjuvants defined in paragraph 23.

29. The method according to paragraph 35 or 36, wherein the nucleic acids are administered intraarterially, intraveneously, or by the routes defined in paragraph 24.

30. The method according to any one of paragraphs 27-29, which includes at least one administration of the nucleic acids per year, such as at least 2, at least 3, at least 4, at least 6, and at least 12 administrations per year.

31. The method according to paragraph 21, wherein presentation to the immune system is effected by administering a non-pathogenic microorganism or virus which is carrying a nucleic acid fragment which encodes and expresses the analogue.

32. The method according to paragraph 31, wherein the virus is a non-virulent pox virus such as a vaccinia virus.

33. The method according to paragraph 31, wherein the microorganism is a bacterium.

34. The method according to any one of paragraphs 31-33, wherein the non-pathogenic microorganism or virus is administered one single time to the animal.

35. A composition for inducing production of antibodies against a multimeric protein, the composition comprising

an immunogenic analogue according to any one of paragraphs 1-17, and

a pharmaceutically and immunologically acceptable carrier and/or vehicle and/or adjuvant.

36. A composition for inducing production of antibodies against a multimeric protein, the composition comprising

a nucleic acid fragment according to paragraph 19, and

a pharmaceutically and immunologically acceptable carrier and/or vehicle and/or adjuvant.

37. The composition according to paragraph 35 or 36, wherein the analogue us formulated as defined in any one of paragraphs 22 or 23.

38. A method for the preparation of the analogue according to any one of paragraphs 1-17, the method comprising culturing a host cell transformed with the nucleic acid fragment according to paragraph 19 under conditions that facilitate expression of the nucleic acid fragment of paragraph 19 and subsequently recovering the analogue as a protein expression product from the culture.

39. The method according to paragraph 38, wherein the host cell is a bacterial host cell.

40. The method according to paragraph 39, wherein the analogue is a soluble expression product.

1. An immunogenic analogue of a human TNFα, wherein said analogue
a) includes substantial fragments of at least 2 monomeric units of human TNFα, wherein said substantial fragments are joined via peptide bonds through a peptide linker,
b) includes at least one MHC Class II binding amino acid sequence that is heterologous to human TNFα, and
c) can be produced as one single expression product from a cell harbouring an expression vector encoding the analogue.

2. The immunogenic analogue according to claim 1, wherein each of the substantial fragments displays a substantial fraction of B-cell epitopes found in the corresponding monomers when being part of the human TNFα protein.

3. The immunogenic analogue according to claim 2, wherein each of the substantial fragments displays essentially all B-cell epitopes found in the corresponding monomers when being part of human TNFα.

4. The immunogenic analogue according to claim 2, wherein an amino acid sequence derived from a monomeric unit is modified by means of amino acid insertion, substitution, deletion or addition so as to reduce toxicity of the analogue as compared to human TNFα and/or so as to introduce the MHC Class II binding amino acid sequence.

5. The immunogenic analogue according to claim 1, wherein each of the substantial fractions comprises essentially the complete amino acid sequence of each monomeric unit, either as a continuous sequence or as a sequence including inserts.

6. The immunogenic analogue according to claim 1, wherein amino acid sequences of all monomeric units of human TNFα are represented in the analogue.

7. The immunogenic analogue according to claim 6 that includes the complete amino acid sequences of the monomers constituting human TNFα, either as unbroken sequences or as sequences including inserts.

8. The immunogenic analogue according to claim 1, wherein the peptide linker includes or contributes to the presence in the analogue of at least one MHC Class II binding amino acid sequence that is heterologous to human TNFα.

9. The immunogenic analogue according to claim 1, wherein the peptide linker is free of and does not contribute to the presence of an MHC Class II binding amino acid sequence in humans.

10. The immunogenic analogue according to claim 1, wherein the MHC Class II binding amino acid sequence binds a majority of MHC Class II molecules in humans.

11. The immunogenic analogue according to claim 1, wherein the at least one MHC Class II binding amino acid sequence is selected from a natural T-cell epitope and an artificial MHC-II binding peptide sequence.

12. The immunogenic analogue according to claim 10, wherein the natural T-cell epitope is selected from a Tetanus toxoid epitope such as P2 or P30, a diphtheria toxoid epitope, an influenza virus hemagluttinin epitope, and a P. falciparum CS epitope.

13. The immunogenic analogue according to claim 1, wherein the 3-dimensional structure of the complete human TNFα is essentially preserved.

14. The immunogenic analogue according to claim 1, wherein the analogue is selected from the group consisting of
two or three complete TNFα monomers joined end-to-end by a peptide linker, wherein at least one peptide linker includes at least one MHC Class II binding amino acid sequence,
two or three complete TNFα monomers joined end-to-end by an inert peptide linker, wherein at least one of the monomers include at least one foreign MHC Class II binding amino acid sequence or wherein at least one foreign MHC Class II binding amino acids sequence is fused to the N- or C-terminal monomer, optionally via an inert linker.

15. An immunogenic analogue of human TNFα, wherein the analogue includes at least one foreign MHC Class II binding amino acid sequence and further has the characteristic of being
a human TNFα monomer or an analogue according to any one of the preceding claims, wherein has been inserted or in-substituted at least one foreign MHC Class II binding amino acid sequence into flexible loop 3, and/or
a human TNFα monomer or an analogue according to any one of the preceding claims, wherein has been introduced at least one disulfide bridge that stabilises the TNFα monomer 3D structure, and/or
a human TNFα monomer or an analogue according to any one of the preceding claims, wherein any one of amino acids 1, 2, 3, 4, 5, 6, 7, 8, and 9 in the amino terminus have been deleted, and/or
a human TNFα monomer or an analogue according to any one of the preceding claims, wherein an inserted or in-substituted at least one foreign MHC Class II binding amino acid sequence into loop 1 in an intron position, and/or
a human TNFα monomer or an analogue according to any one of the preceding claims, wherein at least one foreign MHC Class II binding amino acid sequence is introduced as part of an artificial stalk region in the N-terminus of human TNFα, and/or
a human TNFα monomer or an analogue according to any one of the preceding claims, wherein at least one foreign MHC Class II binding amino acid sequence is introduced so as to stabilize the monomer structure by increasing the hydrophobicity of the trimeric interaction interface, and/or
a human TNFα monomer or an analogue according to any one of the preceding claims, wherein at least one foreign MHC Class II binding amino acid sequence flanked by glycine residues is inserted or in-substituted in the TNFα amino acid sequence, and/or
a human TNFα monomer or an analogue according to any one of the preceding claims, wherein at least one foreign MHC Class II binding amino acid sequence is inserted or in-substituted in the D-E loop, and/or
a human TNFα monomer or an analogue according to any one of the preceding claims, wherein at least one foreign MHC Class II binding amino acid sequence is inserted or in-substituted between two identical subsequences of human TNFα, and/or
a human TNFα monomer or an analogue according to any one of the preceding claims, wherein at least one salt bridge in human TNFα has been strengthened or substituted with a disulphide bridge, and/or
a human TNFα monomer or an analogue according to any one of the preceding claims, wherein solubility and/or stability towards proteolysis is enhanced by introducing mutations that mimic murine TNFα crystalline structure, and/or
a human TNFα monomer or an analogue according to any one of the preceding claims, wherein potential toxicity is reduced or abolished by introduction of at least one point mutation.

16. An immunogenic analogue according to claim 14 or 15, wherein the amino acid sequence of the analogue is selected from the group consisting of SEQ ID NO: 18, 23, 24, 25, 26, 27, 28, 29, 30, 31, 32, 33, 34, 35, 36, 37, 38, 39, 40, 41, 42, 43, 44, 45, 46, 47, 49, 51, 53, 55, 57, and 59, and any amino acid sequence that only include conservative amino acid changes thereof.

17. An immunogenic analogue according to claim 1 or 15 which can be expressed as a soluble protein from bacterial cells.

18. A nucleic acid fragment that encodes an immunogenic analogue according to claim 1 or 15, or a nucleic acid fragment complementary thereto.

19. The nucleic acid fragment according to claim 18 that is a DNA fragment.

20. The nucleic acid fragment according to claim 18 which comprises a nucleic acid sequence selected from the group consisting of SEQ ID NO: 17, 48, 50, 52, 54, 56, and 58 or a nucleic acid sequence complementary thereto.

21. A method for down-regulating a human TNFα in a human, the method comprising effecting presentation to the immune system of an immunogenically effective amount of at least one immunogenic analogue according to claim 1 or an immunogenic analogue of human TNFα, wherein the analogue includes at least one foreign MHC Class II binding amino acid sequence and further has the characteristic of being
a human TNFα monomer or an analogue according to any one of the preceding claims, wherein has been inserted or in-substituted at least one foreign MHC Class II binding amino acid sequence into flexible loop 3, and/or
a human TNFα monomer or an analogue according to any one of the preceding claims, wherein has been introduced at least one disulfide bridge that stabilises the TNFα0 monomer 3D structure, and/or
a human TNFα monomer or an analogue according to any one of the preceding claims, wherein any one of amino acids 1, 2, 3, 4, 5, 6, 7, 8, and 9 in the amino terminus have been deleted, and/or
a human TNFα monomer or an analogue according to any one of the preceding claims, wherein an inserted or in-substituted at least one foreign MHC Class II binding amino acid sequence into loop 1 in an intron position, and/or
a human TNFα monomer or an analogue according to any one of the preceding claims, wherein at least one foreign MHC Class II binding amino acid sequence is introduced as part of an artificial stalk region in the N-terminus of human TNFα, and/or
a human TNFα monomer or an analogue according to any one of the preceding claims, wherein at least one foreign MHC Class II binding amino acid sequence is introduced so as to stabilize the monomer structure by increasing the hydrophobicity of the trimeric interaction interface, and/or
a human TNFα monomer or an analogue according to any one of the preceding claims, wherein at least one foreign MHC Class II binding amino acid sequence flanked by glycine residues is inserted or in-substituted in the TNFα amino acid sequence, and/or
a human TNFα monomer or an analogue according to any one of the preceding claims, wherein at least one foreign MHC Class II binding amino acid sequence is inserted or in-substituted in the D-E loop, and/or
a human TNFα monomer or an analogue according to any one of the preceding claims, wherein at least one foreign MHC Class II binding amino acid sequence is inserted or in-substituted between two identical subsequences of human TNFα, and/or
a human TNFα monomer or an analogue according to any one of the preceding claims, wherein at least one salt bridge in human TNFα has been strengthened or substituted with a disulphide bridge, and/or
a human TNFα monomer or an analogue according to any one of the preceding claims, wherein solubility and/or stability towards proteolysis is enhanced by introducing mutations that mimic murine TNFα crystalline structure, and/or
a human TNFα monomer or an analogue according to any one of the preceding claims, wherein potential toxicity is reduced or abolished by introduction of at least one point mutation.

22. The method according to claim 21, wherein presentation is effected by administering the immunogenic analogue to the autologous host, optionally in admixture with an adjuvant.

23. The method according to claim 22, wherein the adjuvant is selected from the group consisting of an immune targeting adjuvant; an immune modulating adjuvant such as a toxin, a cytokine and a mycobacterial derivative; an oil formulation; a polymer; a micelle forming adjuvant; a saponin; an immunostimulating complex matrix (an ISCOM matrix); a particle; DDA; aluminium adjuvants; DNA adjuvants; γ-inulin; and an encapsulating adjuvant.

24. The method according to claim 21, wherein an immunogenically effective amount of analogue is administered to the animal via a route selected from the parenteral route such as the intradermal, the subdermal, and the intramuscular routes; the peritoneal route; the oral route; the buccal route; the sublinqual route; the epidural route; the spinal route; the anal route; and the intracranial route.

25. The method according to claim 24, wherein the effective amount is between 0.5 μg and 2,000 μg.

26. The method according to claim 24, which includes at least one administration per year, such as at least 2, at least 3, at least 4, at least 6, and at least 12 administrations per year.

27. The method according to claim 21, wherein presentation of the analogue to the immune system is effected by introducing nucleic acid(s) encoding the analogue into the animal's cells and thereby obtaining in vivo expression by the cells of the nucleic acid(s) introduced.

28. The method according to claim 27, wherein the nucleic acid(s) introduced is/are selected from naked DNA, DNA formulated with charged or uncharged lipids, DNA formulated in liposomes, DNA included in a viral vector, DNA formulated with a transfection-facilitating protein or polypeptide, DNA formulated with a targeting protein or polypeptide, DNA formulated with Calcium precipitating agents, DNA coupled to an inert carrier molecule, DNA encapsulated in chitin or chitosan, and DNA formulated with an adjuvant such as the adjuvants defined in claim 23.

29. The method according to claim 27, wherein the nucleic acids are administered intraarterially, intraveneously, or by the routes defined in claim 24.

30. The method according to claim 27, which includes at least one administration of the nucleic acids per year, such as at least 2, at least 3, at least 4, at least 6, and at least 12 administrations per year.

31. The method according to claim 21, wherein presentation to the immune system is effected by administering a non-pathogenic microorganism or virus which is carrying a nucleic acid fragment which encodes and expresses the analogue.

32. The method according to claim 31, wherein the virus is a non-virulent pox virus such as a vaccinia virus.

33. The method according to claim 31, wherein the microorganism is a bacterium.

34. The method according to claim 31, wherein the non-pathogenic microorganism or virus is administered one single time to the animal.

35. A composition for inducing production of antibodies against a multimeric protein, the composition comprising
an immunogenic analogue according to claim 1 or 15, and
a pharmaceutically and immunologically acceptable carrier and/or vehicle and/or adjuvant.

36. A composition for inducing production of antibodies against a multimeric protein, the composition comprising
a nucleic acid fragment according to claim 19, and
a pharmaceutically and immunologically acceptable carrier and/or vehicle and/or adjuvant.

37. The composition according to claim 35 or 36, wherein the analogue is optionally in admixture with an adjuvant, wherein the adjuvant is selected from the group consisting of an immune targeting adjuvant; an immune modulating adjuvant such as a toxin, a cytokine and a mycobacterial derivative; an oil formulation; a polymer; a micelle forming adjuvant; a saponin; an immunostimulating complex matrix (an ISCOM matrix); a particle; DDA; aluminium adjuvants; DNA adjuvants; γ-inulin; and an encapsulating adjuvant.

38. A method for the preparation of the analogue according to claim 1 or 15, the method comprising culturing a host cell transformed with a nucleic acid fragment that encodes an immunogenic analogue according to claim 1 or 15, or a nucleic acid fragment complementary thereto, under conditions that facilitate expression of the nucleic acid fragment, and subsequently recovering the analogue as a protein expression product from the culture.

39. The method according to claim 38, wherein the host cell is a bacterial host cell.

40. The method according to claim 39, wherein the analogue is a soluble expression product.


SEQUENCE LISTING (60 sequences)

SEQ ID NO: 1 - protein, 115 aa, homo sapiens
IPTEIPTSALVKETLALLSTHRTLLIANETLRIPVPVHKNHQLCTEEIFQGIGTLESQTVQGGTVERLFKNLSLIKKYIDGQKKKCGEERRRVNQFLDYLQEFLGVMNTEWIIES

SEQ ID NO: 2 - DNA, 45 bp, Artificial sequence
  Tetanus toxoid P2 epitope
cagtacatcaaagctaactccaaattcatcggcatcaccgaactg

SEQ ID NO: 3 - protein, 15 aa, Artificial sequence
  Tetanus toxoid P2 epitope
QYIKANSKFIGITEL

SEQ ID NO: 4 - DNA, 63 bp, Artificial sequence
  Tetanus toxoid P30 epitope
ttcaacaacttcaccgtttccttctggctgcgcgttccaaaagtttccgcttcccacctggaa

SEQ ID NO: 5 - protein, 21 aa, Artificial sequence
  Tetanus toxoid P30 epitope
FNNFTVSFWLRVPKVSASHLE

SEQ ID NO: 6 - DNA, 39 bp, Artificial sequence
  Pan DR binding peptide (PADRE)
gccaagttcgtggccgcttggaccctgaaggccgcagct

SEQ ID NO: 7 - protein, 13 aa, Artificial sequence
  Pan DR binding peptide (PADRE)
AKFVAAWTLKAAA

SEQ ID NO: 8 - DNA, 858 bp, Artificial sequence
  2 human IL5 monomers joined by P30 and P2 epitopes
atgaggatgcttctgcatttgagtttgctggctcttggagctgcctacgtgtatgccatccccacagaaattcccacaagtgcattggtgaaagagaccttggcactgctttctactcatcgaactctgctgatagccaatgagactctgaggattcctgttcctgtacataaaaatcaccaactgtgcactgaagaaatctttcagggaataggcacactggagagtcaaactgtgcaagggggtactgtggaaagactattcaaaaacttgtccttaataaagaaatacattgacggccaaaaaaaaaagtgtggagaagaaagacggagagtaaaccaattcctagactacctgcaagagtttcttggtgtaatgaacaccgagtggataatagaaagtttcaacaacttcaccgtgagcttctggctgcgcgtgcctaaggtgagcgccagccacctggagcagtacatcaaggccaactccaagttcatcggcatcaccgagctgatccccacagaaattcccacaagtgcattggtgaaagagaccttggcactgctttctactcatcgaactctgctgatagccaatgagactctgaggattcctgttcctgtacataaaaatcaccaactgtgcactgaagaaatctttcagggaataggcacactggagagtcaaactgtgcaagggggtactgtggaaagactattcaaaaacttgtccttaataaagaaatacattgacggccaaaaaaaaaagtgtggagaagaaagacggagagtaaaccaattcctagactacctgcaagagtttcttggtgtaatgaacaccgagtggataatagaaagttga

SEQ ID NO: 9 - protein, 285 aa, Artificial sequence
  2 human IL5 monomers joined by P30 and P2 epitopes
MRMLLHLSLLALGAAYVYAIPTEIPTSALVKETLALLSTHRTLLIANETLRIPVPVHKNHQLCTEEIFQGIGTLESQTVQGGTVERLFKNLSLIKKYIDGQKKKCGEERRRVNQFLDYLQEFLGVMNTEWIIESFNNFTVSFWLRVPKVSASHLEQYIKANSKFIGITELIPTEIPTSALVKETLALLSTHRTLLIANETLRIPVPVHKNHQLCTEEIFQGIGTLESQTVQGGTVERLFKNLSLIKKYIDGQKKKCGEERRRVNQFLDYLQEFLGVMNTEWIIES

SEQ ID NO: 10 - DNA, 858 bp, Artificial sequence
  2 human IL5 monomers joined by P2 and P30 epitopes
atgaggatgcttctgcatttgagtttgctggctcttggagctgcctacgtgtatgccatccccacagaaattcccacaagtgcattggtgaaagagaccttggcactgctttctactcatcgaactctgctgatagccaatgagactctgaggattcctgttcctgtacataaaaatcaccaactgtgcactgaagaaatctttcagggaataggcacactggagagtcaaactgtgcaagggggtactgtggaaagactattcaaaaacttgtccttaataaagaaatacattgacggccaaaaaaaaaagtgtggagaagaaagacggagagtaaaccaattcctagactacctgcaagagtttcttggtgtaatgaacaccgagtggataatagaaagtcagtacatcaaggccaactccaagttcatcggcatcaccgagctgttcaacaacttcaccgtgagcttctggctgcgcgtgcctaaggtgagcgccagccacctggagatccccacagaaattcccacaagtgcattggtgaaagagaccttggcactgctttctactcatcgaactctgctgatagccaatgagactctgaggattcctgttcctgtacataaaaatcaccaactgtgcactgaagaaatctttcagggaataggcacactggagagtcaaactgtgcaagggggtactgtggaaagactattcaaaaacttgtccttaataaagaaatacattgacggccaaaaaaaaaagtgtggagaagaaagacggagagtaaaccaattcctagactacctgcaagagtttcttggtgtaatgaacaccgagtggataatagaaagttga

SEQ ID NO: 11 - protein, 285 aa, Artificial sequence [sequence omitted]

SEQ ID NO: 12 - DNA, 864 bp, Artificial sequence
  Two human IL5 monomers joined by diglycine linker and including terminally positioned P30 and P2 epitopes
atgaggatgcttctgcatttgagtttgctggctcttggagctgcctacgtgtatgccatccccacagaattcaacaacttcaccgtgagcttctggctgcgcgtgcctaaggtgagcgccagccacctggagattcccacaagtgcattggtgaaagagaccttggcactgctttctactcatcgaactctgctgatagccaatgagactctgaggattcctgttcctgtacataaaaatcaccaactgtgcactgaagaaatctttcagggaataggcacactggagagtcaaactgtgcaagggggtactgtggaaagactattcaaaaacttgtccttaataaagaaatacattgacggccaaaaaaaaaagtgtggagaagaaagacggagagtaaaccaattcctagactacctgcaagagtttcttggtgtaatgaacaccgagtggataatagaaagtggcggtatccccacagaaattcccacaagtgcattggtgaaagagaccttggcactgctttctactcatcgaactctgctgatagccaatgagactctgaggattcctgttcctgtacataaaaatcaccaactgtgcactgaagaaatctttcagggaataggcacactggagagtcaaactgtgcaagggggtactgtggaaagactattcaaaaacttgtccttaataaagaaatacattgacggccaaaaaaaaaagtgtggagaagaaagacggagagtaaaccaattcctagactacctgcaagagtttcttggtgtaatgaacaccgagtggataatagaaagtcagtacatcaaggccaactccaagttcatcggcatcaccgagctgtga

SEQ ID NO: 13 - protein, 287 aa, Artificial sequence [sequence omitted]

SEQ ID NO: 14 - DNA, 864 bp, Artificial sequence
  Two human IL5 monomers joined by a di-glycine linker and including terminally positioned tetanus toxoid P2 and P30 epitopes
atgaggatgcttctgcatttgagtttgctggctcttggagctgcctacgtgtatgccatccccacagaacagtacatcaaggccaactccaagttcatcggcatcaccgagctgattcccacaagtgcattggtgaaagagaccttggcactgctttctactcatcgaactctgctgatagccaatgagactctgaggattcctgttcctgtacataaaaatcaccaactgtgcactgaagaaatctttcagggaataggcacactggagagtcaaactgtgcaagggggtactgtggaaagactattcaaaaacttgtccttaataaagaaatacattgacggccaaaaaaaaaagtgtggagaagaaagacggagagtaaaccaattcctagactacctgcaagagtttcttggtgtaatgaacaccgagtggataatagaaagtggcggtatccccacagaaattcccacaagtgcattggtgaaagagaccttggcactgctttctactcatcgaactctgctgatagccaatgagactctgaggattcctgttcctgtacataaaaatcaccaactgtgcactgaagaaatctttcagggaataggcacactggagagtcaaactgtgcaagggggtactgtggaaagactattcaaaaacttgtccttaataaagaaatacattgacggccaaaaaaaaaagtgtggagaagaaagacggagagtaaaccaattcctagactacctgcaagagtttcttggtgtaatgaacaccgagtggataatagaaagtttcaacaacttcaccgtgagcttctggctgcgcgtgcctaaggtgagcgccagccacctggagtga

SEQ ID NO: 15 - protein, 287 aa, Artificial sequence [sequence omitted]

SEQ ID NO: 16 - DNA, 477 bp, Artificial sequence
  Human wt TNF (codons optimised)
atggtgcgctcaagctcgcgcacgccgagtgacaaaccagtagctcatgttgtggccaaccctcaggcggaaggccagctccaatggttaaatcgtcgcgcgaacgccctgctggcgaacggcgtggaactgcgtgataaccagctggtggtccccagcgaggggctgtatctgatctattcacaggtgttgtttaagggtcagggttgtccgagcacccacgttctgctgacgcataccatttctcgtattgctgtatcttatcaaactaaagtcaatttactttcggcgatcaaatccccgtgccaacgtgagacccctgaaggagcggaagccaaaccttggtacgaaccgatctatctggggggcgtttttcagctcgaaaaaggtgatcggctgagcgccgaaattaatcgcccggactaccttgatttcgcagagtccggtcaggtctacttcggcattatcgcattgtaa

SEQ ID NO: 17 - protein, 158 aa, Artificial sequence
  Human wt TNF (codons optimised)
MVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 18 - protein, 170 aa, Artificial sequence
  hTNF with inserted PADRE
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAKFVAAWTLKAAAAEAKPWYEPIYLGGVFQLEKGDRLSAEINRPDYLDFAESGQVYFGIIAL

SEQ ID NO: 19 - DNA, 39 bp, Artificial sequence
  Pan DR binding peptide (PADRE)
gcgaagttcgttgcagcttggaccctgaaggccgctgca

SEQ ID NO: 20 - protein, 13 aa, Artificial sequence
  Pan DR binding peptide (PADRE)
AKFVAAWTLKAAA

SEQ ID NO: 21 - DNA, 1437 bp, Artificial sequence
  Monomeric mimic of trimeric human TNF
atggtgcgcagcagcagccgcacccccagcgacaagcccgtggcccacgtggtggccaacccccaggccgagggccaactgcagtggctgaaccgccgcgccaacgccctgctggcaaacggcgtggagctgcgcgacaaccagctggtggtgcccagcgagggcctgtacctgatctacagccaggtgctgttcaagggccagggctgccccagcacccacgtgctgctgacccacaccatcagccgcatcgccgtgagctaccagaccaaggtgaacctgctgagcgccatcaagagcccctgccagcgcgagacccccgagggcgccgaggccaagccctggtacgagcccatctacctcggcggcgtgttccagctggagaagggcgaccgcctgagcgccgagatcaaccgccccgactacctggacttcgccgagagcggccaggtgtacttcggcatcatcgccctgggtggcggagtccggtcctcctcccggacaccatccgacaaaccagtcgctcatgtcgtcgctaatccacaagctgaaggtcaacttcaatggcttaatcggcgggctaatgctcttcttgctaatggtgtcgaacttcgggacaatcaacttgtcgtcccatccgaaggtctttatcttatttattcccaagtcctttttaaaggtcaaggttgtccatccacacatgtccttcttacacatacaatttcccggattgctgtctcctatcaaacaaaagtcaatcttctttccgctattaaatccccatgtcaacgggaaacaccagaaggtgctgaagctaaaccttggtatgaaccaatttatcttggtggtgtctttcaacttgaaaaaggtgaccggctttccgctgaaattaatcggccagattatcttgactttgctgaatccggtcaagtctattttggtattattgctctgggcggtggggttcgttcttcttctcgtacgccgtctgataagccggttgcgcacgttgttgcgaacccgcaggcggaggggcaattgcagtggttgaatcgtcgtgcgaacgcgttgttggcgaatggggttgaattgcgtgataaccaattggttgttccgtctgaggggttgtacttgatatattctcaggttttgttcaaagggcaagggtgcccgtctacgcatgttttgttgacgcacacgatatctcgtatagcggtttcttaccagacgaaggttaatttgttgtctgcgataaaatctccgtgtcaacgtgaaacgccggaaggggcggaggcgaagccgtggtatgaaccgatatacttggggggggtttttcagttggaaaaaggggatcgtttgtctgcggagataaaccgtccggactatttggatttcgcggaatctgggcaagtttactttgggataatagcgctgtaa

SEQ ID NO: 22 - protein, 478 aa, Artificial sequence
  Monomeric mimic of trimeric human TNF
MVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 23 - protein, 170 aa, Artificial sequence
  hTNF with inserted PADRE
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPAKFVAAWTLKAAAEGAEAKPWYEPIYLGGVFQLEKGDRLSAEINRPDYLDFAESGQVYFGIIAL

SEQ ID NO: 24 - protein, 170 aa, Artificial sequence
  hTNF with inserted PADRE
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEAKFVAAWTLKAAAGAEAKPWYEPIYLGGVFQLEKGDRLSAEINRPDYLDFAESGQVYFGIIAL

SEQ ID NO: 25 - protein, 169 aa, Artificial sequence
  hTNF with 12 amino acids of PADRE inserted
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAKFVAAWTLKAAAEAKPWYEPIYLGGVFQLEKGDRLSAEINRPDYLDFAESGQVYFGIIAL

SEQ ID NO: 26 - protein, 167 aa, Artificial sequence
  hTNF with PADRE substituted in
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAKFVAAWTLKAAAKPWYEPIYLGGVFQLEKGDRLSAEINRPDYLDFAESGQVYFGIIAL

SEQ ID NO: 27 - protein, 165 aa, Artificial sequence
  hTNF with in-substituted PADRE
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPAKFVAAWTLKAAAKPWYEPIYLGGVFQLEKGDRLSAEINRPDYLDFAESGQVYFGIIAL

SEQ ID NO: 28 - protein, 173 aa, Artificial sequence
  hTNF with C-terminal tri-glycine linker and PADRE
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGAKFVAAWTLKAAA

SEQ ID NO: 29 - protein, 170 aa, Artificial sequence
  hTNF with inserted PADRE and additional disulfide bridge
VRSSSRTPSDKPVAHVVANPQAEGQLQWLNRRANALLANGVELRDNQLVVPSEGLYLIYSQVLFKGCGCPSTHVLLTHTISRIAVSYQTKVNLLSAIKSPCQRETPEGAKFVAAWTLKAAAAECKPWYEPIYLGGVFQLEKGDRLSAEINRPDYLDFAESGQVYFGIIAL

SEQ ID NO: 30 - protein, 170 aa, Artificial sequence
  hTNF with inserted PADRE and additional disulphide bridge
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSCIKSPCQRETPEGAKFVAAWTLKAAAAEAKPWYEPCYLGGVFQLEKGDRLSAEINRPDYLDFAESGQVYFGIIAL

SEQ ID NO: 31 - protein, 162 aa, Artificial sequence
  hTNF truncate with inserted PADRE
SDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAKFVAAWTLKAAAAEAKPWYEPIYLGGVFQLEKGDRLSAEINRPDYLDFAESGQVYFGIIAL

SEQ ID NO: 32 - protein, 169 aa, Artificial sequence
  hTNF with PADRE inserted
VRSSSRTPSDKPVAHVVAKFVAAWTLKAAANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 33 - protein, 165 aa, Artificial sequence
  hTNF with in-substituted PADRE
VRSSSRTPSDKPVAHVVAKFVAAWTLKAAAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 34 - protein, 170 aa, Artificial sequence
  hTNF with added artificial stalk region and inserted PADRE
MAKFVAAWTLKAAARSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 35 - protein, 170 aa, Artificial sequence
  hTNF with inserted PADRE and single stabilising mutation
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAKFVAAWTLKAAAAEAKPWYEPIYLGGVFQLEKGDRLSAEINRPDYLDFAESGQVYFGIIAF

SEQ ID NO: 36 - protein, 170 aa, Artificial sequence
  hTNF with inserted PADRE and one single mutation
VRSSSRTPSDKPVAHVVANPQAEGQLQWLNRRANALLANGVELRDNQLFVPSEGLYLIYSQVLFKGQGCPSTHVLLTHTISRIAVSYQTKVNLLSAIKSPCQRETPEGAKFVAAWTLKAAAAEAKPWYEPIYLGGVFQLEKGDRLSAEINRPDYLDFAESGQVYFGIIAL

SEQ ID NO: 37 - protein, 174 aa, Artificial sequence
  hTNF with inserted glycine-linked PADRE
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGGGAKFVAAWTLKAAAGGAEAKPWYEPIYLGGVFQLEKGDRLSAEINRPDYLDFAESGQVYFGIIAL

SEQ ID NO: 38 - protein, 167 aa, Artificial sequence
  hTNF with in-substituted PADRE
VRSSSRTPSDKPVAHVVANPQAEGQLQWLNRRANALLANGVELRDNQLVVPSEGLYLIYSQVLFKGQGCPSTHVLLTHTISRIAKFVAAWTLKAAAYQTKVNLLSAIKSPCQRETPEGAEAKPWYEPIYLGGVFQLEKGDRLSAEINRPDYLDFAESGQVYFGIIAL

SEQ ID NO: 39 - protein, 157 aa, Artificial sequence
  hTNF with in-substituted PADRE
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLAKFVAAWTLKAAAESGQVYFGIIAL

SEQ ID NO: 40 - protein, 160 aa, Artificial sequence
  hTNF with in-substituted PADRE
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEAKFVAAWTLKAAAESGQVYFGIIAL

SEQ ID NO: 41 - protein, 157 aa, Artificial sequence
  hTNF with insubstituted PADRE
VRSSSRTPSDKPVAHVVANPQAEGQLQWLNRRANALLANGVELRDNQLVVPSEGLYLIYSQVLAKFVAAWTLKAAATHTISRIAVSYQTKVNLLSAIKSPCQRETPEGAEAKPWYEPIYLGGVFQLEKGDRLSAEINRPDYLDFAESGQVYFGIIAL

SEQ ID NO: 42 - protein, 157 aa, Artificial sequence
  hTNF with in-substituted PADRE
VRSSSRTPSDKPVAHVVANPQAEGQLQWLNRRANALLANGVELRDNQLVVPSEGLYLIYSQVLFKGQGCPSAKFVAAWTLKAAAVSYQTKVNLLSAIKSPCQRETPEGAEAKPWYEPIYLGGVFQLEKGDRLSAEINRPDYLDFAESGQVYFGIIAL

SEQ ID NO: 43 - protein, 157 aa, Artificial sequence
  hTNF with insubstituted PADRE
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLAKFVAAWTLKAAADYLDFAESGQVYFGIIAL

SEQ ID NO: 44 - protein, 176 aa, Artificial sequence
  hTNF with inserted peptide and duplication of 6 amino acids
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAKFVAAWTLKAAARETPEGAEAKPWYEPIYLGGVFQLEKGDRLSAEINRPDYLDFAESGQVYFGIIAL

SEQ ID NO: 45 - protein, 174 aa, Artificial sequence
  hTNF with inserted PADRE and duplication of 4 amino acids
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAKFVAAWTLKAAATPEGAEAKPWYEPIYLGGVFQLEKGDRLSAEINRPDYLDFAESGQVYFGIIAL

SEQ ID NO: 46 - protein, 194 aa, Artificial sequence
  hTNF with inserted tetanus toxoid P2 and P30 epitopes
MVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGQYIKANSKFIGITELFNNFTVSFWLRVPKVSASHLEAEAKPWYEPIYLGGVFQLEKGDRLSAEINRPDYLDFAESGQVYFGIIAL

SEQ ID NO: 47 - protein, 194 aa, Artificial sequence
  hTNF with inserted tetanus toxoid P2 and P30 epitopes
MVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGFNNFTVSFWLRVPKVSASHLEQYIKANSKFIGITELAEAKPWYEPIYLGGVFQLEKGDRLSAEINRPDYLDFAESGQVYFGIIAL

SEQ ID NO: 48 - DNA, 1545 bp, Artificial sequence
  3 hTNF sequences joined by glycince linkers and tetanus toxoid P2 and P30 epitopes
atggtgcgcagcagcagccgcacccccagcgacaagcccgtggcccacgtggtggccaacccccaggccgagggccaactgcagtggctgaaccgccgcgccaacgccctgctggcaaacggcgtggagctgcgcgacaaccagctggtggtgcccagcgagggcctgtacctgatctacagccaggtgctgttcaagggccagggctgccccagcacccacgtgctgctgacccacaccatcagccgcatcgccgtgagctaccagaccaaggtgaacctgctgagcgccatcaagagcccctgccagcgcgagacccccgagggcgccgaggccaagccctggtacgagcccatctacctcggcggcgtgttccagctggagaagggcgaccgcctgagcgccgagatcaaccgccccgactacctggacttcgccgagagcggccaggtgtacttcggcatcatcgccctgggtggcggacagtacatcaaagctaactccaaattcatcggcatcaccgaactggtccggtcctcctcccggacaccatccgacaaaccagtcgctcatgtcgtcgctaatccacaagctgaaggtcaacttcaatggcttaatcggcgggctaatgctcttcttgctaatggtgtcgaacttcgggacaatcaacttgtcgtcccatccgaaggtctttatcttatttattcccaagtcctttttaaaggtcaaggttgtccatccacacatgtccttcttacacatacaatttcccggattgctgtctcctatcaaacaaaagtcaatcttctttccgctattaaatccccatgtcaacgggaaacaccagaaggtgctgaagctaaaccttggtatgaaccaatttatcttggtggtgtctttcaacttgaaaaaggtgaccggctttccgctgaaattaatcggccagattatcttgactttgctgaatccggtcaagtctattttggtattattgctctgggcggtgggttcaacaacttcaccgtttccttctggctgcgcgttccaaaagtttccgcttcccacctggaagttcgttcttcttctcgtacgccgtctgataagccggttgcgcacgttgttgcgaacccgcaggcggaggggcaattgcagtggttgaatcgtcgtgcgaacgcgttgttggcgaatggggttgaattgcgtgataaccaattggttgttccgtctgaggggttgtacttgatatattctcaggttttgttcaaagggcaagggtgcccgtctacgcatgttttgttgacgcacacgatatctcgtatagcggtttcttaccagacgaaggttaatttgttgtctgcgataaaatctccgtgtcaacgtgaaacgccggaaggggcggaggcgaagccgtggtatgaaccgatatacttggggggggtttttcagttggaaaaaggggatcgtttgtctgcggagataaaccgtccggactatttggatttcgcggaatctgggcaagtttactttgggataatagcgctgtaa

SEQ ID NO: 49 - protein, 514 aa, Artificial sequence
  3 hTNF sequences joined by glycince linkers and tetanus toxoid P2 and P30 epitopes
MVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGQYIKANSKFIGITELVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGFNNFTVSFWLRVPKVSASHLEVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 50 - DNA, 1545 bp, Artificial sequence
  3 hTNF monomers joined by tri-glycine linkers and tetanus toxoid P2 and P30 epitopes
atggtgcgcagcagcagccgcacccccagcgacaagcccgtggcccacgtggtggccaacccccaggccgagggccaactgcagtggctgaaccgccgcgccaacgccctgctggcaaacggcgtggagctgcgcgacaaccagctggtggtgcccagcgagggcctgtacctgatctacagccaggtgctgttcaagggccagggctgccccagcacccacgtgctgctgacccacaccatcagccgcatcgccgtgagctaccagaccaaggtgaacctgctgagcgccatcaagagcccctgccagcgcgagacccccgagggcgccgaggccaagccctggtacgagcccatctacctcggcggcgtgttccagctggagaagggcgaccgcctgagcgccgagatcaaccgccccgactacctggacttcgccgagagcggccaggtgtacttcggcatcatcgccctgggtggcggattcaacaacttcaccgtttccttctggctgcgcgttccaaaagtttccgcttcccacctggaagtccggtcctcctcccggacaccatccgacaaaccagtcgctcatgtcgtcgctaatccacaagctgaaggtcaacttcaatggcttaatcggcgggctaatgctcttcttgctaatggtgtcgaacttcgggacaatcaacttgtcgtcccatccgaaggtctttatcttatttattcccaagtcctttttaaaggtcaaggttgtccatccacacatgtccttcttacacatacaatttcccggattgctgtctcctatcaaacaaaagtcaatcttctttccgctattaaatccccatgtcaacgggaaacaccagaaggtgctgaagctaaaccttggtatgaaccaatttatcttggtggtgtctttcaacttgaaaaaggtgaccggctttccgctgaaattaatcggccagattatcttgactttgctgaatccggtcaagtctattttggtattattgctctgggcggtgggcagtacatcaaagctaactccaaattcatcggcatcaccgaactggttcgttcttcttctcgtacgccgtctgataagccggttgcgcacgttgttgcgaacccgcaggcggaggggcaattgcagtggttgaatcgtcgtgcgaacgcgttgttggcgaatggggttgaattgcgtgataaccaattggttgttccgtctgaggggttgtacttgatatattctcaggttttgttcaaagggcaagggtgcccgtctacgcatgttttgttgacgcacacgatatctcgtatagcggtttcttaccagacgaaggttaatttgttgtctgcgataaaatctccgtgtcaacgtgaaacgccggaaggggcggaggcgaagccgtggtatgaaccgatatacttggggggggtttttcagttggaaaaaggggatcgtttgtctgcggagataaaccgtccggactatttggatttcgcggaatctgggcaagtttactttgggataatagcgctgtaa

SEQ ID NO: 51 - protein, 514 aa, Artificial sequence
  3 hTNF monomers joined by tri-glycine linkers and tetanus toxoid P2 and P30 epitopes
MVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGFNNFTVSFWLRVPKVSASHLEVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGQYIKANSKFIGITELVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 52 - DNA, 1554 bp, Artificial sequence
  3 hTNF monomers joined by tri-glycine linkers and with P2 and P30 epitopes introduced
atggtgcgcagcagcagccgcacccccagcgacaagcccgtggcccacgtggtggccaacccccaggccgagggccaactgcagtggctgaaccgccgcgccaacgccctgctggcaaacggcgtggagctgcgcgacaaccagctggtggtgcccagcgagggcctgtacctgatctacagccaggtgctgttcaagggccagggctgccccagcacccacgtgctgctgacccacaccatcagccgcatcgccgtgagctaccagaccaaggtgaacctgctgagcgccatcaagagcccctgccagcgcgagacccccgagggcgccgaggccaagccctggtacgagcccatctacctcggcggcgtgttccagctggagaagggcgaccgcctgagcgccgagatcaaccgccccgactacctggacttcgccgagagcggccaggtgtacttcggcatcatcgccctgggtggcggagtccggtcctcctcccggacaccatccgacaaaccagtcgctcatgtcgtcgctaatccacaagctgaaggtcaacttcaatggcttaatcggcgggctaatgctcttcttgctaatggtgtcgaacttcgggacaatcaacttgtcgtcccatccgaaggtctttatcttatttattcccaagtcctttttaaaggtcaaggttgtccatccacacatgtccttcttacacatacaatttcccggattgctgtctcctatcaaacaaaagtcaatcttctttccgctattaaatccccatgtcaacgggaaacaccagaaggtgctgaagctaaaccttggtatgaaccaatttatcttggtggtgtctttcaacttgaaaaaggtgaccggctttccgctgaaattaatcggccagattatcttgactttgctgaatccggtcaagtctattttggtattattgctctgggcggtgggcagtacatcaaagctaactccaaattcatcggcatcaccgaactggttcgttcttcttctcgtacgccgtctgataagccggttgcgcacgttgttgcgaacccgcaggcggaggggcaattgcagtggttgaatcgtcgtgcgaacgcgttgttggcgaatggggttgaattgcgtgataaccaattggttgttccgtctgaggggttgtacttgatatattctcaggttttgttcaaagggcaagggtgcccgtctacgcatgttttgttgacgcacacgatatctcgtatagcggtttcttaccagacgaaggttaatttgttgtctgcgataaaatctccgtgtcaacgtgaaacgccggaaggggcggaggcgaagccgtggtatgaaccgatatacttggggggggtttttcagttggaaaaaggggatcgtttgtctgcggagataaaccgtccggactatttggatttcgcggaatctgggcaagtttactttgggataatagcgctgggtggcggattcaacaacttcaccgtttccttctggctgcgcgttccaaaagtttccgcttcccacctggaataa

SEQ ID NO: 53 - protein, 517 aa, Artificial sequence
  3 hTNF monomers joined by tri-glycine linkers and with P2 and P30 epitopes introduced
MVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGQYIKANSKFIGITELVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGFNNFTVSFWLRVPKVSASHLE

SEQ ID NO: 54 - DNA, 1485 bp, Artificial sequence
  3 hTNF joined by tri-glycine linkers and PADRE added C-terminally
atggtgcgcagcagcagccgcacccccagcgacaagcccgtggcccacgtggtggccaacccccaggccgagggccaactgcagtggctgaaccgccgcgccaacgccctgctggcaaacggcgtggagctgcgcgacaaccagctggtggtgcccagcgagggcctgtacctgatctacagccaggtgctgttcaagggccagggctgccccagcacccacgtgctgctgacccacaccatcagccgcatcgccgtgagctaccagaccaaggtgaacctgctgagcgccatcaagagcccctgccagcgcgagacccccgagggcgccgaggccaagccctggtacgagcccatctacctcggcggcgtgttccagctggagaagggcgaccgcctgagcgccgagatcaaccgccccgactacctggacttcgccgagagcggccaggtgtacttcggcatcatcgccctgggtggcggagtccggtcctcctcccggacaccatccgacaaaccagtcgctcatgtcgtcgctaatccacaagctgaaggtcaacttcaatggcttaatcggcgggctaatgctcttcttgctaatggtgtcgaacttcgggacaatcaacttgtcgtcccatccgaaggtctttatcttatttattcccaagtcctttttaaaggtcaaggttgtccatccacacatgtccttcttacacatacaatttcccggattgctgtctcctatcaaacaaaagtcaatcttctttccgctattaaatccccatgtcaacgggaaacaccagaaggtgctgaagctaaaccttggtatgaaccaatttatcttggtggtgtctttcaacttgaaaaaggtgaccggctttccgctgaaattaatcggccagattatcttgactttgctgaatccggtcaagtctattttggtattattgctctgggcggtggggttcgttcttcttctcgtacgccgtctgataagccggttgcgcacgttgttgcgaacccgcaggcggaggggcaattgcagtggttgaatcgtcgtgcgaacgcgttgttggcgaatggggttgaattgcgtgataaccaattggttgttccgtctgaggggttgtacttgatatattctcaggttttgttcaaagggcaagggtgcccgtctacgcatgttttgttgacgcacacgatatctcgtatagcggtttcttaccagacgaaggttaatttgttgtctgcgataaaatctccgtgtcaacgtgaaacgccggaaggggcggaggcgaagccgtggtatgaaccgatatacttggggggggtttttcagttggaaaaaggggatcgtttgtctgcggagataaaccgtccggactatttggatttcgcggaatctgggcaagtttactttgggataatagcgctgggagggggtgccaagttcgtggccgcttggaccctgaaggccgcagcttaa

SEQ ID NO: 55 - protein, 494 aa, Artificial sequence
  3 hTNF joined by tri-glycine linkers and PADRE added C-terminally
MVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGAKFVAAWTLKAAA

SEQ ID NO: 56 - DNA, 1476 bp, Artificial sequence
  3 hTNF joined by tri-glycine linkers and with PADRE in the C-terminus
atggtgcgcagcagcagccgcacccccagcgacaagcccgtggcccacgtggtggccaacccccaggccgagggccaactgcagtggctgaaccgccgcgccaacgccctgctggcaaacggcgtggagctgcgcgacaaccagctggtggtgcccagcgagggcctgtacctgatctacagccaggtgctgttcaagggccagggctgccccagcacccacgtgctgctgacccacaccatcagccgcatcgccgtgagctaccagaccaaggtgaacctgctgagcgccatcaagagcccctgccagcgcgagacccccgagggcgccgaggccaagccctggtacgagcccatctacctcggcggcgtgttccagctggagaagggcgaccgcctgagcgccgagatcaaccgccccgactacctggacttcgccgagagcggccaggtgtacttcggcatcatcgccctgggtggcggagtccggtcctcctcccggacaccatccgacaaaccagtcgctcatgtcgtcgctaatccacaagctgaaggtcaacttcaatggcttaatcggcgggctaatgctcttcttgctaatggtgtcgaacttcgggacaatcaacttgtcgtcccatccgaaggtctttatcttatttattcccaagtcctttttaaaggtcaaggttgtccatccacacatgtccttcttacacatacaatttcccggattgctgtctcctatcaaacaaaagtcaatcttctttccgctattaaatccccatgtcaacgggaaacaccagaaggtgctgaagctaaaccttggtatgaaccaatttatcttggtggtgtctttcaacttgaaaaaggtgaccggctttccgctgaaattaatcggccagattatcttgactttgctgaatccggtcaagtctattttggtattattgctctgggcggtggggttcgttcttcttctcgtacgccgtctgataagccggttgcgcacgttgttgcgaacccgcaggcggaggggcaattgcagtggttgaatcgtcgtgcgaacgcgttgttggcgaatggggttgaattgcgtgataaccaattggttgttccgtctgaggggttgtacttgatatattctcaggttttgttcaaagggcaagggtgcccgtctacgcatgttttgttgacgcacacgatatctcgtatagcggtttcttaccagacgaaggttaatttgttgtctgcgataaaatctccgtgtcaacgtgaaacgccggaaggggcggaggcgaagccgtggtatgaaccgatatacttggggggggtttttcagttggaaaaaggggatcgtttgtctgcggagataaaccgtccggactatttggatttcgcggaatctgggcaagtttactttgggataatagcgctggccaagttcgtggccgcttggaccctgaaggccgcagcttaa

SEQ ID NO: 57 - protein, 491 aa, Artificial sequence
  3 hTNF joined by tri-glycine linkers and with PADRE in the C-terminus
MVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALAKFVAAWTLKAAA

SEQ ID NO: 58 - DNA, 1545 bp, Artificial sequence
  3 hTNF joined by glycine linkers and P2 and P30 introduced
atggtgcgcagcagcagccgcacccccagcgacaagcccgtggcccacgtggtggccaacccccaggccgagggccaactgcagtggctgaaccgccgcgccaacgccctgctggcaaacggcgtggagctgcgcgacaaccagctggtggtgcccagcgagggcctgtacctgatctacagccaggtgctgttcaagggccagggctgccccagcacccacgtgctgctgacccacaccatcagccgcatcgccgtgagctaccagaccaaggtgaacctgctgagcgccatcaagagcccctgccagcgcgagacccccgagggcgccgaggccaagccctggtacgagcccatctacctcggcggcgtgttccagctggagaagggcgaccgcctgagcgccgagatcaaccgccccgactacctggacttcgccgagagcggccaggtgtacttcggcatcatcgccctgggtggcggagtccggtcctcctcccggacaccatccgacaaaccagtcgctcatgtcgtcgctaatccacaagctgaaggtcaacttcaatggcttaatcggcgggctaatgctcttcttgctaatggtgtcgaacttcgggacaatcaacttgtcgtcccatccgaaggtctttatcttatttattcccaagtcctttttaaaggtcaaggttgtccatccacacatgtccttcttacacatacaatttcccggattgctgtctcctatcaaacaaaagtcaatcttctttccgctattaaatccccatgtcaacgggaaacaccagaaggtgctgaagctaaaccttggtatgaaccaatttatcttggtggtgtctttcaacttgaaaaaggtgaccggctttccgctgaaattaatcggccagattatcttgactttgctgaatccggtcaagtctattttggtattattgctctgggcggtgggcagtacatcaaagctaactccaaattcatcggcatcaccgaactggttcgttcttcttctcgtacgccgtctgataagccggttgcgcacgttgttgcgaacccgcaggcggaggggcaattgcagtggttgaatcgtcgtgcgaacgcgttgttggcgaatggggttgaattgcgtgataaccaattggttgttccgtctgaggggttgtacttgatatattctcaggttttgttcaaagggcaagggtgcccgtctacgcatgttttgttgacgcacacgatatctcgtatagcggtttcttaccagacgaaggttaatttgttgtctgcgataaaatctccgtgtcaacgtgaaacgccggaaggggcggaggcgaagccgtggtatgaaccgatatacttggggggggtttttcagttggaaaaaggggatcgtttgtctgcggagataaaccgtccggactatttggatttcgcggaatctgggcaagtttactttgggataatagcgctgttcaacaacttcaccgtttccttctggctgcgcgttccaaaagtttccgcttcccacctggaataa

SEQ ID NO: 59 - protein, 514 aa, Artificial sequence
  3 hTNF joined by glycine linkers and P2 and P30 introduced
MVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGQYIKANSKFIGITELVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALFNNFTVSFWLRVPKVSASHLE

SEQ ID NO: 60 - DNA, 2773 bp, Artificial sequence
  p2ZOp2F expression vector for insect cells
ggatcatgatgataaacaatgtatggtgctaatgttgcttcaacaacaattctgttgaactgtgttttcatgtttgccaacaagcacctttatactcggtggcctccccaccaccaacttttttgcactgcaaaaaaacacgcttttgcacgcgggcccatacatagtacaaactctacgtttcgtagactattttacataaatagtctacaccgttgtatacgctccaaatacactaccacacattgaacctttttgcagtgcaaaaaagtacgtgtcggcagtcacgtaggccggccttatcgggtcgcgtcctgtcacgtacgaatcacattatcggaccggacgagtgttgtcttatcgtgacaggacgccagcttcctgtgttgctaaccgcagccggacgcaactccttatcggaacaggacgcgcctccatatcagccgcgcgttatctcatgcgcgtgaccggacacgaggcgcccgtcccgcttatcgcgcctataaatacagcccgcaacgatctggtaaacacagttgaacagcatctgttcgaatttaaagcttggtaccctcgagctcagctgaattctggatcctctagaccggtcatatgcggccgcggatcgatcgatatctgactaaatcttagtttgtattgtcatgttttaatacaatatgttatgtttaaatatgtttttaataaattttataaaataatttcaacttttattgtaacaacattgtccatttacacactcctttcaagcgcgtgggactcgatgctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgatgataaacaatgtatggtgctaatgttgcttcaacaacaattctgttgaactgtgttttcatgtttgccaacaagcacctttatactcggtggcctccccaccaccaacttttttgcactgcaaaaaaacacgcttttgcacgcgggcccatacatagtacaaactctacgtttcgtagactattttacataaatagtctacaccgttgtatacgctccaaatacactaccacacattgaacctttttgcagtgcaaaaaagtacgtgtcggcagtcacgtaggccggccttatcgggtcgcgtcctgtcacgtacgaatcacattatcggaccggacgagtgttgtcttatcgtgacaggacgccagcttcctgtgttgctaaccgcagccggacgcaactccttatcggaacaggacgcgcctccatatcagccgcgcgttatctcatgcgcgtgaccggacacgaggcgcccgtcccgcttatcgcgcctataaatacagcccgcaacgatctggtaaacacagttgaacagcatctgttcgaattaattcggatctctgcagcacgtgttgacaattaatcatcggcatagtatatcggcatagtataatacgactcactataggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgaccgacgccgaccaacaccgccggtccgacggcggcccacgggtcccaggggggtcgacctcgaaacttgtttattgcagcttataatggttacaaataaagcaatagcatcacaaatttcacaaataaagcatttttttcactgcattctagttgtggtttgtccaaactcatcaatgtatcttatcatgtct